(19) United States
(12) Patent Application Publication
(10) Pub. No.: US 2022/0304923 A1
(43) Pub. Date: Sep. 29, 2022

(54) USE OF ORAL CHEW FOR MODULATING ORAL MICROBIOTA AND IMPROVING ORAL HEALTH

(71) Applicant: MARS, INCORPORATED, McLean, VA (US)

(72) Inventors: Lucy Jane HOLCOMBE, Melton Mowbray, Leicestershire (GB); Corryn Victoria WALLIS, Melton Mowbray, Leicestershire (GB); Avika Kishorlal RUPARELL, Melton Mowbray, Leicestershire (GB); Philip Martin MCGENITY, Melton Mowbray, Leicestershire (GB)

(73) Assignee: MARS, INCORPORATED, McLean, VA (US)

(21) Appl. No.: 17/616,873

(22) PCT Filed: Jun. 8, 2020
(86) PCT No.: PCT/US2020/036712

§ 371 Date: Dec. 6, 2021

(30) Foreign Application Priority Data
Jun. 6, 2019 (GB) 1908109.0

(51) Int. Cl. A61K 9/00; A23K 50/42; A61P 1/02

(52) U.S. Cl. CPC A61K 9/0056; A23K 50/42; A61P 1/02

(57) ABSTRACT

Disclosed herein is the use of an oral chew and related methods for modulating the canine oral microbiota. The method can include administering an oral chew to a dog in an amount effective to improve the oral health of the dog.

### CROSS-REFERENCE TO RELATED APPLICATION

This application claims priority to UK Patent Application No. 1908109.0, filed on Jun. 6, 2019, the contents of which are incorporated herein by reference in its entirety.

### FIELD

The presently disclosed subject matter relates to the use of an oral chew and related methods for modulating the oral microbiota in animals, and in particular the canine oral microbiota.

### SEQUENCE LISTING

The instant application contains a Sequence Listing which has been submitted electronically in ASCII format and is hereby incorporated by reference in its entirety. Said ASCII copy, created on Jun. 8, 2020, is named 0692690413.txt and is 15,244 bytes in size.

### BACKGROUND

Periodontal disease describes a spectrum of deteriorating conditions affecting the supporting tissues around the teeth. Clinical signs vary from redness and inflammation of the gingiva (gingivitis) to destruction of the tissues that support the teeth, in some cases leading to tooth loss (periodontitis). Presentation with the disease in small animal practice is frequent (Lund et al. 1999, O. Neill et al. 2014), and figures suggest that between 44% and 100% of the canine pet population are affected (Gad 1968, Harvey et al. 1994, Hoffmann and Gaengler 1996, Kyllar and Witter 2005, Kortegaard et al. 2008).

The development of periodontal disease follows a multifactorial hypothesis, where microbial contributors play a fundamental role via dental plaque. As such, scientific investigations have elucidated several theories regarding the role of bacteria in the initiation of pathogenesis. Regardless of the precise mechanism, however, it is through disturbances to the equilibrium of the oral ecosystem and associated bacterial community that initiates the disease cascade. Understanding fluctuations in microbiota is now possible due to advancements in molecular sequencing and bioinformatics technologies which allow a holistic perspective of canine oral plaque bacteria (Riggio et al. 2011, Davis et al. 2013, Wallis et al. 2015). These advancements have not only delivered the ability to perform comparative analyses across different time points, for example, in the disease's progression, but additionally have aided the understanding of bacterial associations between health states. Significant insights in this area to date have been provided by comprehensive in vivo studies. A cross-sectional survey of plaque bacterial species from 223 client owned dogs with healthy gingiva, gingivitis or mild periodontitis found health was associated with Gram negative genera including Bergeyella, Moraxella and Porphyromonas while mild periodontitis was associated with the Gram positive genera Actinomyces, Peptostreptococcus and Peptostreptococcaceae (Davis et al. 2013). In a longitudinal study focusing on miniature schnauzers, Wallis et al. 2015 collected subgingival plaque samples every six weeks for a period up to 60 weeks. With the progression to mild periodontitis, they observed a reduction in the abundance of particular Gram negative species, namely Bergeyella zoohelcum COT-186, Moraxella sp. COT-017, Pasteurellaceae sp. COT-080, and Neisseria shayeganii COT-090.

Prevention of periodontal disease is undoubtedly preferred than the necessity for treatment. Strategies including tooth brushing and the use of regular oral care chews aim to maintain a healthy homeostasis through restricting plaque (and dental calculus) growth to low levels (Gorrel and Bierer 1999, Gorrel et al. 1999, Brown and McGenity 2005, Hennet et al. 2006, Clarke et al. 2011, Quest 2013, Harvey et al. 2015).

The maintenance of oral health in animals is important to maintain the overall health of the animal. Pet foods and chews exist that offer some benefit to the oral health of animals. Such a pet food is described in EP Patent No. 0575021A2. A pet food type described therein is also commercially available (Hill's Prescription Diet@ t/d). The pet food claims to help keep a dog's teeth clean. The food has a nutritionally balanced mix of carbohydrate, protein, fat, vitamins and minerals. The cleaning action of the food is stated as stemming from the expanded striated structural matrix, which is designed to fracture when chewed by a dog and so offer a mechanical cleaning action via abrasive contact between the separated matrix layers and the tooth. The chew described in PCT Appln. Pub. No. WO2014/155113 can also be used to help keep a dog's teeth clean. Although such products can aid in the maintaining of the oral health of animals, there still exists a need to provide improved means for maintaining the oral health of animals and, in particular, for modulating the oral microbiota.

The presently disclosed subject matter has advantageously identified that feeding a particular diet to animals including an oral chew as disclosed herein can have a significant effect on their oral microbiota in order to promote a healthier microbiota and reduce the likelihood of the development of oral disease or disorder (e.g., periodontal disease).

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages, and in accordance with the purpose of the disclosed subject matter, as embodied and broadly described, the disclosed subject matter includes in one aspect an oral chew for modulating the canine oral microbiota.

The present disclosure provides such a means in the form of an edible oral chew that modulates the oral microbiota of a dog when the chew is consumed.

A chew can be differentiated from food by virtue of its nutritional content. Specifically, a conventional dog ‘food’ is nutritionally complete and provides the full range of the dog's daily nutrition requirements. It is also intended to be the major source of the dog's calorific intake. A chew need not provide such nutrition or calorific content. Normally a chew will be formulated in such that, unlike a main meal food, it would not be suitable to be the dog's only source of nutrition.

A chew can further be distinguished from a ‘food’ with regard to its size. The largest pieces in a food product are generally smaller than the size of a chew. For instance, PCT Appln. Pub. No. WO01/050882A discloses a food product which is reported as having a large size compared to other dried pet food, and discloses several examples. The largest of these examples is a triangular kibble having the following dimensions: thickness 16 mm, base 28 mm and sides 32 mm. This is in contrast to a dog chew which is significantly larger. The oral chew of the present disclosure can be an individual piece having a largest dimension of at least about 40 mm, at least, at least about 60 mm, or at least about 70 mm. The size of the oral chew can vary in size according to the size of dog to which it is to be given. For example, chews for toy and small breeds can be smaller than chews for medium, large and giant breeds. Chews for larger breeds can be more than 100 mm or even more than 120 mm in length.

A chew can be further distinguished with regard to the time taken to consume a piece of chew compared to a piece of food. Normally the consumption time for a piece of chew is much longer than a piece of food. A piece of food can generally be consumed in less than 10 seconds by an average sized dog, whereas a chew would take at least 20 seconds for an average-sized dog to consume. In one embodiment, a chew of the present disclosure would typically take at least 90 seconds, more typically at least 120 seconds for an average-sized dog to consume. The chew of the present disclosure can exhibit a lasting time (in seconds) per gram of chew of at least 3 seconds per gram of chew.

The starch content of the chew of the present disclosure can be from 10% to 80% or from 40 wt % to 70 wt % relative to the total weight of the chew. As used herein, weights relative to the total weight of the chew are with respect to the finished product, which is ready to be consumed by a dog. The starch content can be from 45 wt % to 65 wt % relative to the total weight of the chew, or 45 wt % to 70 wt %, or 50 wt % to 65 wt %, or 50 wt % to 60 wt % relative to the total weight of the chew. The relatively high starch content of the chew of the present disclosure contributes to the chew's ability to retain its shape.

In one embodiment, the chew of the disclosure comprises a starch content of about 40 wt % to about 70 wt % relative to the total weight of the chew; a humectant content of 5 wt/c to 20 wt % relative to the total weight of the chew; wherein the chew has a density of about 1.0 g cm⁻³ or less.

The chew of the present disclosure can have a texture that, when consumed by a dog, contributes to maintaining the dog's oral health.

The chew can exhibit a characteristic spongy texture, for example due to its composition (and particularly the humectant content, which confers the ability to retain water), and a relatively low density. This texture allows the chew of the present disclosure to elastically rebound to a certain extent and so at least partially close up a hole formed by a dog tooth after the dog tooth is withdrawn during the chewing process. In other words, the chew of the present disclosure demonstrates an ability to self-heal to a certain extent.

The oral chew can be any appropriate oral chew for administration to a dog. For example the chew can be an oral chew as defined in PCT Appln. Pub. No. WO2014/155113.

The oral microbiota can be modulated by increasing or decreasing the presence or prevalence of particular microorganisms, particularly bacterial species or groups of bacterial species. For example, the oral microbiota can be modulated by increasing the number of bacterial species associated or strongly associated with good oral health in a dog to which the chew is administered, compared to the expected microbiota in that dog, if the chew was not administered. The oral microbiota can also be modulated by increasing the prevalence, i.e., the number present, of one or more bacterial species associated with good oral health, particularly one or more bacterial species strongly associated with good oral health. The oral microbiota can also be modulated by increasing the ratio of bacteria or bacterial species associated with good oral health to bacteria or bacterial species associated with poor oral health in the oral microbiota.

Bacterial species associated or strongly associated with good oral health are known in the art. Examples of bacterial species associated with or strongly associated with good oral health are shown in FIG. 3. Modulating the oral microbiota can comprise increasing the prevalence of one, two, three, four, five or more of Prevotella sp. COT-282, Propionibacterium sp. COT-296, Catonella sp. COT-257, Peptostreptococcaceae bacterium FOT-054 and Corynebacterium mustelae.

The oral microbiota can be modulated by decreasing the number of bacterial species associated with poor oral health or with disease, particularly species strongly associated with poor oral health or disease in a dog to which the chew is administered, compared to the expected microbiota in that dog, if the chew was not administered. The oral microbiota can also be modulated by decreasing the prevalence or relative proportion, i.e., the number present, of one or more bacterial species associated with poor oral health or disease, particularly one or more bacterial species strongly associated with poor oral health or disease.

Bacterial species associated or strongly associated with poor oral health or disease are known in the art. Examples of bacterial species associated with or strongly associated with poor oral health or disease are shown in FIG. 3. Modulating the oral microbiota can comprise decreasing the prevalence of one, two, three, four, five or more of Fretibacterium sp. FOT-218, Neisseria canis, Anaerovorax sp. COT-125, Peptostreptococcaceae bacterium COT-030, Pelistega sp. COT-267, Bacteroidia bacterium COT-387, Desulfomicrobium orale and Helococcus sp. FOT-023.

In certain aspects, the oral chew can be for administration daily, every other day, weekly or fortnightly.

In certain aspects, the oral chew can be for administration for at least 5, 8, 10, 13, 15, 18, 20, 23, 25, 28, 35 or 42 times.

In one embodiment, the chew is for administration to a dog with a clean mouth, e.g., after it has received a scale and polish, or a dog with generally healthy gingiva. Without being bound by the theory, the chew is thought to encourage the colonization of the clean mouth with more bacteria not associated with periodontal disease compared to a clean mouth to which a chew is not administered. In one embodiment, the colonization can be decreased with poor oral health or disease-related bacteria.

The dog can be any breed of dog, including toy, small, medium, large and giant breeds. In one embodiment, the dog is a medium, large or giant breed. In one embodiment, the dog is a medium breed. In certain embodiments, the dog is a toy or small breed.

Modulating the oral microbiota can result in improved oral health, for example, by reducing the likelihood of the dog developing periodontal disease.

A further aspect of the present disclosure is an oral chew for improving the oral health of a dog, wherein the oral health is improved by modulation of the oral microbiota.

A further aspect of the present disclosure provides the use of an oral chew for improving oral health in a dog, wherein the oral health is improved by modulation of the oral microbiota.

A further aspect of the present disclosure provides a method of modulating the oral microbiota in a dog, comprising the step of feeding the dog an oral chew.

The step of feeding the dog the chew can be carried out, for example, 5, 8, 10, 13, 15, 18, 20, 23, 25, 28, 35, 42 or more times. The chew can be fed to the dog daily, twice weekly, weekly or fortnightly. The chew can be fed to the dog for a period of 3 days, a year, for the life of the dog.

The dog can be a dog with a clean mouth, that is to say a dog that has received a scale and polish, or that has had its teeth cleaned, or that has generally healthy gingiva.

The method can also comprise the step of cleaning the dog's teeth or mouth.

A further aspect of the present disclosure provides a method for improving the oral health of a dog by modulating the dog's oral microbiota, comprising the step of feeding the dog an oral chew.

The step of feeding the dog the chew can be carried out, for example 5, 8, 10, 13, 15, 18, 20, 23, 25, 28 or more times. The chew can be fed to the dog daily, twice weekly, weekly or fortnightly. The chew can be fed to the dog for a period of 3 days, a year, for the life of the dog.

Features of the aspects of the present disclosure can be as defined in relation to the first listed aspect of the present disclosure.

In certain aspects, the present disclosure provides a method of modulating an oral microbiota in a dog. The method includes administering an oral chew in an amount effective to improve the oral health of the dog.

In certain embodiments, the dog can be administered the oral chew daily, twice weekly, weekly or fortnightly. In certain embodiments, the dog is administered the oral chew over a period of 3 days a year, for the life of the dog. In particular aspects, the dog is administered the oral chew at least 5, 8, 10, 13, 15, 18, 20, 23, 25, 28, 35 or 42 times.

In certain aspects, the method can further include cleaning the mouth of the dog prior to administering the oral chew. In particular aspects, the mouth of the dog can be cleaned prior to administering any oral chew.

In certain embodiments, the dog can be from a medium, large or giant breed.

In certain aspects, the oral microbiota can be modulated by increasing the number of bacterial species associated with good oral health, or the prevalence or relative proportion of bacteria from bacterial species associated with good oral health, compared to an expected microbiota. In particular, modulation of the oral microbiota can include increasing the prevalence of at least one of Prevotella sp. COT-282, Propionibacterium sp. COT-296, Catonella sp. COT-257, Peptostreptococcaceae bacterium FOT-054 and Corynebacterium mustelae.

In other aspects, the oral microbiota can be modulated by decreasing the number of bacterial species associated with poor oral health or with disease, or the prevalence or relative proportion of bacteria from bacterial species associated with poor oral health or disease, compared to an expected microbiota. In particular, modulation of the oral microbiota can include decreasing the prevalence of at least one of Fretibacterium sp. FOT-218, Neisseria canis, Anaerovorax sp. COT-125, Peptostreptococcaceae bacterium COT-030, Pelistega sp. COT-267, Bacteroidia bacterium COT-387, Desulfomicrobium orale and Helococcus sp. FOT-023.

In certain aspects, the improved oral health of the dog is a reduction in periodontal disease or oral malodour.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the kits and methods of the disclosed subject matter. Together with the description, the drawings serve to explain the principles of the disclosed subject matter.

### BRIEF SUMMARY OF THE DRAWINGS

The present disclosure will now be described in detail, by way of example only, with reference to the drawings, in which:

FIG. 1 depicts principal component scores from analysis performed on the log₁₀ proportions of all OTUs identified in the study described in Example 1: start pre-test phase (red), end pre-test phase (blue), end of 28 day test phase chew (yellow) and no chew (purple).

FIG. 2 depicts taxonomic composition per phase in accordance with Example 1. Phylogenetic distribution of operational taxonomic units (OTUs) based on sequence read counts across the supragingival plaque samples. Asterisks indicate candidate phyla.

FIG. 3 depicts average proportions with 95% confidence intervals for the OTUs which showed a significant difference and odds ratio >2 between chew (yellow) and no chew (purple) in accordance with Example 1.

FIG. 4 depicts Shannon diversity index for the phase groups in accordance with Example 1: start pre-test phase (red), end pre-test phase (blue), end of 28 day test phase chew (yellow) and no chew (purple).

FIG. 5 is a combined proportion plot, with confidence intervals, of the species associated with health and with disease in accordance with Example 1.

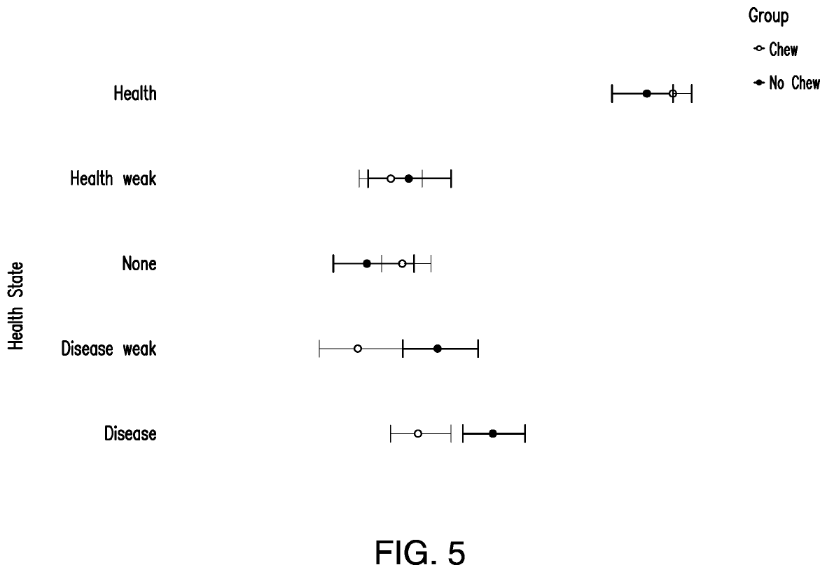

FIG. 6 is a bar chart based on only taxa numbers per health state for the chew and no chew groups in accordance with Example 1.

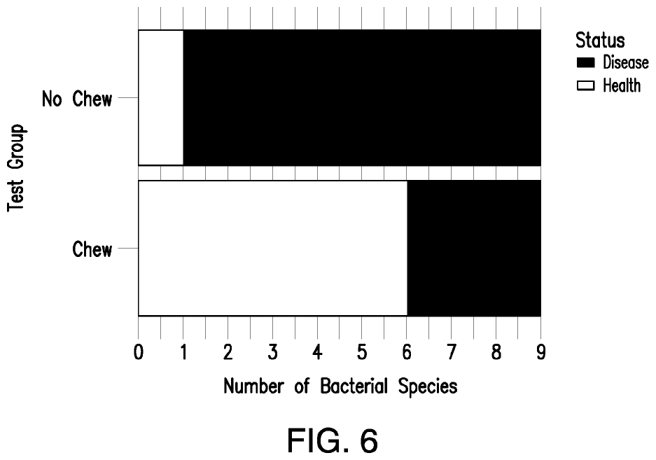

FIG. 7 depicts the foldchange in bacterial species found in the chew and no chew groups in accordance with Example 1.

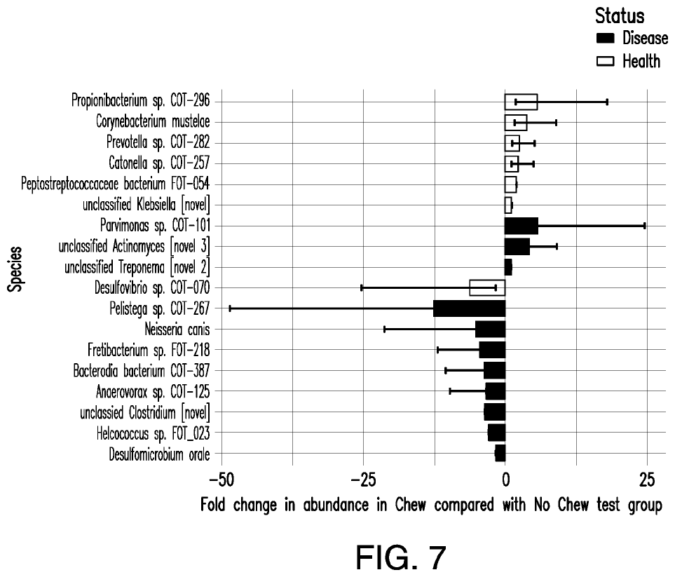

FIG. 8 depicts the means and 95% confidence intervals for COT-030 Cq normalized to UniB Cq relative to nothing of qPCR results for two dietary regimes: (2) commercially available main meal diet+oral care chew B (daily feed treat); and (3) commercially available main meal diet alone (control) in accordance with Example 2.

### DETAILED DESCRIPTION

Reference will now be made in detail to the various exemplary embodiments of the disclosed subject matter, exemplary embodiments of which are illustrated in the accompanying drawings. The presently disclosed subject matter relates to the use of an oral chew and related methods for modulating the canine oral microbiota. Such modulation can advantageously provide an improvement in oral health of the dog, e.g., a reduction in periodontal disease, plaque, calculus, or stain.

### A. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the methods and compositions of the present disclosure and how to make and use them.

Throughout the description and claims of this specification, the words “comprise” and “contain” and variations of the words, for example “comprising” and “comprises,” mean “including but not limited to,” and do not exclude other components, integers or steps. Moreover, the singular encompasses the plural unless the context otherwise requires. In particular, where the indefinite article is used, the specification is to be understood as contemplating plurality as well as singularity, unless the context requires otherwise.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

The term “animal” as used in accordance with the present disclosure refers to a wide variety of animals, such as quadrupeds, primates, and other mammals. For example, the term “animal” can refer to domestic animals including, but not limited to, dogs, cats, horses, cows, ferrets, rabbits, pigs, rats, mice, gerbils, hamsters, goats, and the like. The term “animal” can also refer to wild animals including, but not limited to, bison, elk, deer, venison, duck, fowl, fish, and the like. In some embodiments, the animal is a companion animal. In certain instances, the animal is a domestic dog or cat.

The terms “animal feed,” “animal feed compositions,” “pet food,” “pet food article,” “pet food product”, “edible product” or “pet food composition” are used interchangeably herein and refer to a composition intended for ingestion by an animal or pet. Any composition intended for ingestion by an animal or pet is suitable for use with the present disclosure. Such compositions can include kibble or dry foods, moist or wet foods, semi-moist foods, frozen or freeze-dried foods, raw foods, or combinations thereof. Compositions of the present disclosure can be used, for example, as a main meal, a meal supplement, a treat, or a combination thereof. The compositions can be nutritionally balanced. In alternate embodiments, the compositions are not nutritionally balanced. For example, and not by way of limitation, pet foods can include, without limitation, nutritionally balanced compositions suitable for daily feed, such as kibbles, as well as supplements and/or treats, which can be nutritionally balanced. In an alternative embodiment, the supplement and/or treats are not nutritionally balanced.

The terms “chew” or “oral chew” refers an edible product that in some instances, in one aspect, can be differentiated from food by virtue of its nutritional content. Specifically, a conventional dog “food” is nutritionally complete and provides the full range of the dog's daily nutrition requirements. It is also intended to be the major source of the dog's calorific intake. A chew need not provide such nutrition or calorific content. A chew can further be distinguished from a “food” with regard to its size. The largest pieces in a food product are generally smaller than the size of a chew. A chew can be further distinguished with regard to the time taken to consume a piece of chew compared to a piece of food. Normally the consumption time for a piece of chew is much longer than a piece of food. For example, and not by way of limitation, the edible chew product can be moulded, aerated or extruded.

The phrase “expected microbiota” can refer to the actual microbiota found before the administration of the edible product. In one embodiment, the expected microbiota can refer to the actual microbiota found before the administration of the edible product and before any method has been used to clean the animal's mouth such as a scale and polish. Alternatively, it can refer to the predicted microbiota, based on microbiota found in other animals of the same or similar species or breeds.

The phrase “modulating the oral microbiota” refers to causing the oral microbiota population to change, compared to the oral microbiota that would be expected to be found if the animal had not been fed the edible product of the present disclosure. Modulation of the oral microbiota can comprise promoting health-associated oral cavity flora.

The terms “nutritionally balanced” or “nutritionally complete” in reference to a composition means that the composition, such as pet food, has known required nutrients to sustain life in proper amounts and proportion based on recommendations of recognized authorities, including governmental agencies, such as, but not limited to, National Research Council (NRC) and The European Pet Food Industry (FEDIAF) guidelines (e.g., http://www.fediaforg/images/FEDIAF_Nutritional_Guidelines_2019_Update_030519.pdf), in the field of pet nutrition, except for the additional need for water.

As used herein, the term “oral disease or disorder,” refers to a disease or disorder that occurs in an oral cavity of a subject (e.g., an animal) and that is caused by or is associated with one or more bacteria. For example, the disease or disorder can affect the teeth or the gums of the subject. Exemplary oral diseases or disorders of the present disclosure include, but are not limited to, periodontal disease, caries, gingival stomatitis, odontoclastic resorptive lesions, and oral malodor.

The term “oral microbiota” refers to the microorganisms found in the oral cavity. In particular, it can refer to the bacteria found in the oral cavity, and more specifically to bacterial composition of dental plaque or oral biofilms. It can refer to plaque above (supragingival) and/or below the gum line (subgingival), and/or gingival margin plaque, or biofilms present in the mouth such as on the tongue or cheek or bacteria in the saliva.

As used herein, the term “periodontal disease,” also known as gum disease, refers to an inflammation or infection that affect the tissues surrounding the teeth. Periodontal disease can range in severity, e.g., from gingivitis (e.g., dental plaque-induced gingivitis) to periodontitis.

As used herein, and as is well-understood in the art, “treatment” refers to an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. The decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment. The term “preventing,” as used herein, means partially or completing treating before the disorder or condition occurs.

As used herein, the term “weight percent” is meant to refer to the quantity by weight of a constituent or component, for example, in the pet food composition as a percentage of the overall weight of the pet food composition. The terms “weight percent,” “wt-%,” “wt. %”, and “wt %” are used interchangeably.

Preferred features of each aspect of the presently disclosed subject matter can be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, can be taken independently or in any combination. That is, all embodiments and/or features of any embodiment can be combined in any way and/or combination, unless such features are incompatible.

### B. Bacterial in the Oral Microbiota/Microbiome

The present disclosure relates to, inter alia, edible products and related methods for modulating the oral microbiota of an animal. The one or more bacteria modulated can be associated with an oral disease or disorder, e.g., periodontal disease, or good oral health. The animal can be a companion animal, such as a domestic dog or a cat. In particular aspects, the companion animal is a domestic dog. The dog can be any breed of dog, including toy, small, medium, large and giant breeds. In certain embodiments, the dog is a medium, large or giant breed. In particular embodiments, the dog is a medium breed. In certain embodiments, the dog is a toy or small breed.

In some embodiments, the one or more bacteria associated with periodontal disease can be Peptostreptococcaceae sp. In some embodiments, the one or more bacteria associated with periodontal disease is selected from the group consisting of Peptostreptococcus sp., Synergistes sp., Clostridiales sp., Eubacterium nodatum, Selenomonas sp., Bacteroidetes sp., Odoribacter denticanis, Desulfomicrobium ovale, Moraxella sp., Bacteroides denticanoris, Fillifactor villosus, Porphyromonas canoris, Porphyromonas gulae, Treponema denticola, or Porphyromonas salivosa. In certain embodiments, the one or more bacteria includes Peptostreptococcus sp., volatile organic compound producing bacteria, or both. In further embodiments, the one or more bacteria includes Peptostreptococcaceae XIII [G-1] sp., Peptostreptococcaceae COT-030, Peptostreptococcaceae COT-005/004, Peptostreptococcaceae COT-047, and/or Peptostreptococcaceae COT-019.

Bacterial community profiles within an oral microbiome of an animal can vary depending on the source of a sample taken from the animal. For example, three discrete oral niches can include soft tissue surfaces, such as the lip, cheek, and tongue; hard tissue surfaces, such as the teeth; and saliva. In some embodiments, the oral niche is from a hard tissue surface, such as one or more teeth. In some embodiments, the oral niche includes the gingival margin or supragingival surface.

In certain embodiments, the oral microbiota can be modulated by increasing or decreasing the presence or prevalence of particular microorganisms, particularly bacterial species or groups of bacterial species. In certain embodiments, for example, and not by way of limitation such bacterial species or groups of bacterial species can include those in Tables 3 and 4. In certain embodiments, the phyla of such species can include Proteobacteria, Actinobacteria, Firmicutes, Bacteroidetes, Saccharibacteria (TM7), Spirochaetaes, Synergistetes, or combinations thereof. The oral microbiota can be modulated by increasing the number of bacterial species associated or strongly associated with good oral health in a dog to which the chew is administered, compared to the expected microbiota in that dog, if the chew was not administered. The oral microbiota can also be modulated by increasing the prevalence, i.e., the number present, of one or more bacterial species associated with good oral health, particularly one or more bacterial species strongly associated with good oral health. The oral microbiota can also be modulated by increasing the ratio of bacteria or bacterial species associated with good oral health to bacteria or bacterial species associated with poor oral health in the oral microbiota.

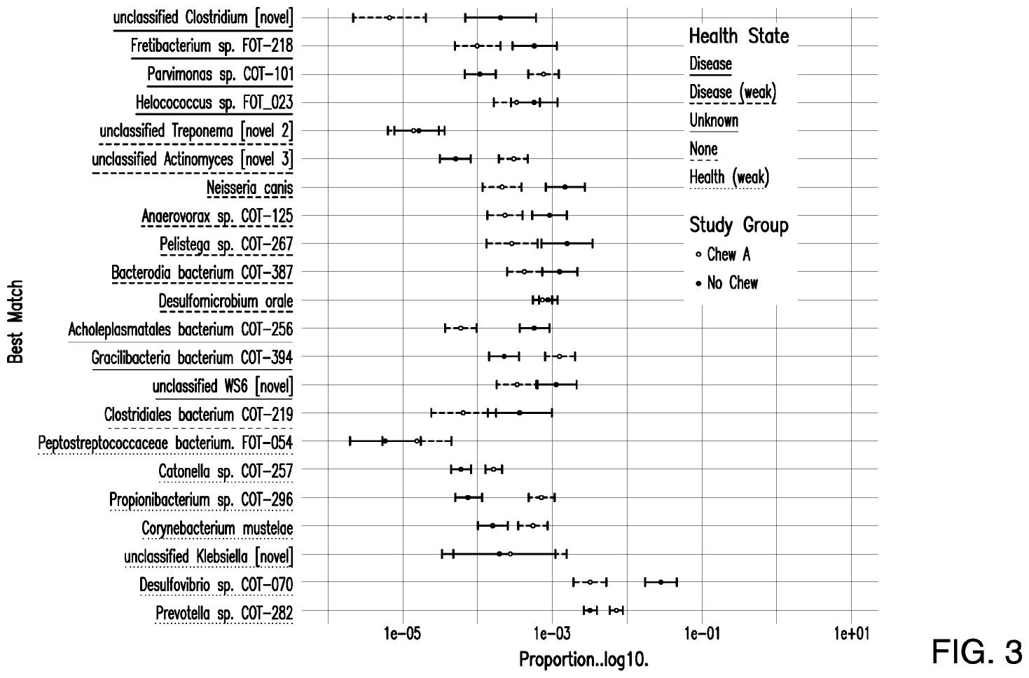

Bacterial species associated or strongly associated with good oral health are known in the art. Examples of bacterial species associated with or strongly associated with good oral health are shown in FIG. 3 and Table 3. For example, any not by way of limitation, bacterial species associated with or strongly associated with good oral health include Prevotella sp. COT-282, Propionibacterium sp. COT-296, Catonella sp. COT-257, Peptostreptococcaceae bacterium FOT-054 and Corynebacterium mustelae. Modulating the oral microbiota can comprise increasing the prevalence of one, two, three, four, five or more of Prevotella sp. COT-282, Propionibacterium sp. COT-296, Catonella sp. COT-257, Peptostreptococcaceae bacterium FOT-054 and Corynebacterium mustelae.

The oral microbiota can be modulated by decreasing the number of bacterial species associated with poor oral health or with disease, particularly species strongly associated with poor oral health or disease in a dog to which the chew is administered, compared to the expected microbiota in that dog, if the chew was not administered. The oral microbiota can also be modulated by decreasing the prevalence or relative proportion, i.e. the number present, of one or more bacterial species associated with poor oral health or disease, particularly one or more bacterial species strongly associated with poor oral health or disease.

Bacterial species associated or strongly associated with poor oral health or disease are known in the art. Examples of bacterial species associated with or strongly associated with poor oral health or disease are shown in FIG. 3 and Table 3. For example, any not by way of limitation, bacterial species associated with or strongly associated with poor oral health include Fretibacterium sp. FOT-218, Neisseria canis, Anaerovorax sp. COT-125, Peptostreptococcaceae bacterium COT-030, Pelistega sp. COT-267, Bacteroidia bacterium COT-387, Desulfomicrobium orale and Helococcus sp. FOT-023. Modulating the oral microbiota can comprise decreasing the prevalence of one, two, three, four, five or more of Fretibacterium sp. FOT-218, Neisseria canis, Anaerovorax sp. COT-125, Peptostreptococcaceae bacterium COT-030, Pelistega sp. COT-267, Bacteroidia bacterium COT-387, Desulfomicrobium orale and Helococcus sp. FOT-023.

### C. Sulfur Containing Amino Acids

Sulfur containing amino acids are amino acids that contribute to the maintenance and integrity of several cellular systems, e.g., cellular redox state, detoxification from free radicals and reactive oxygen species. Sulfur belongs to the same group in the periodic table as oxygen but is much less electronegative. This difference accounts for some distinctive properties of the sulfur containing amino acids. Sulfur containing amino acids are cytotoxic to prokaryotes.

In certain embodiments, the sulfur containing amino acid can be L-amino acid or D-amino acid. In certain embodiments, the sulfur containing amino acid is methionine or a methionine-related amino acid. In certain embodiments, the sulfur containing amino acid is cysteine. In certain embodiments, the sulfur containing amino acid is a cysteine providing derivative. In certain embodiments, the sulfur containing amino acid is homocysteine. In certain embodiments, the sulfur-containing amino acid is taurine. In certain embodiments, the sulfur containing amino acid is N-acetyl cysteine.

In certain embodiments, without limitation, the sulfur containing amino acid can be cysteine sulfinic acid, cysteic acid, homocysteine sulfinic acid, homocysteic acid, serine-O-sulfate, and S-sulfo-cysteine. In certain embodiments, the sulfur-containing amino acid can be a sulfur-containing amino acid derivative. In certain embodiments, the sulfur containing amino acid derivative can be a proteogenic or non-proteogenic amino acid containing a thiol-group, a mercaptan-group, or a thioester-group. Non-limiting examples for sulfur-containing amino acid derivative include S-adenosylmethionine, cystathionine, S-adenosylhomocysteine, glutathione, N-Carbamoyl-L-cysteine, N-Acetylcysteamine, 7-thiomethyl glutamate, 2-amino-A2-thiazoline-4-carboxylic acid, 3-methylthioaspartic acid, 3-thio-L-aspartic acid, S-substituted L-cysteine, D-penicillamine disulfide, L-homolanthionine, L-polyhomomethionine, cystine, dihomomethionine, ergothioneine, hexahomomethionine, hexahomomethionine S-oxide, homocystines, homomethionine, pentahomomethionine, pentahomomethionine S-oxide, tetrahomomethionine, thioproline, and trihomomethionine. Additional non-limiting examples of sulfur containing amino acids include alliin, S-allyl cysteine, S-aminoethyl-L-cysteine, cysteinyldopa, Djenkolic acid, ethionine, felinine, N-formylmethionine, hawkinsin, lanthionine, and lanthionine ketimine.

In certain embodiments, the presently disclosed subject matter contemplates prodrugs of sulfur containing amino acids. For example, but not way of limitation, prodrugs of sulfur containing amino acids can be 2-(polyhydroxy-alkyl)thizolidine-4(R)-carboxylic acid and 2-(polyacetoxyalkyl)thizolidine-4(R)-carboxylic acid.

In certain embodiments, the sulfur containing amino acid of the presently disclosed subject matter is delivered to the animal in an amount from about 0.001 g to about 10 g per 1,000 kcal. For example, but not by way of limitation, the sulfur containing amino acid can be present in the amount of 1 mg to about 10 g, from about 10 mg to about 10 g, from about 100 mg to about 10 g, from about 250 mg to about 10 g, from about 500 mg to about 10 g, from about 750 mg to about 10 g, from about 1 g to about 10 g, and values in between.

### D. Oral Chews

The present disclosure provides for the use of oral chews and related method for modulating the oral microbiota, and in particular the canine oral microbiota. The method can include administering an oral chew to an animal such as a dog in an amount effective to improve the oral health of the animal. Modulating the oral microbiota can result in improved oral health, for example by reducing the likelihood of the animal developing periodontal disease. Further, the improved oral health can include a reduction in periodontal disease, plaque, calculus, or stain.

Oral chews of the present disclosure can include any appropriate oral chew for administration to an animal, such as a dog. For example, and not by way of limitation, the chew can be an oral chew as defined in PCT Appln. Pub. No. WO2014/155113. In certain embodiments, the oral chew of the present disclosure can be moulded, aerated or extruded.

In certain embodiments, oral chews of the present disclosure, in certain aspects, can be differentiated from “food” by virtue of its nutritional content. Specifically, for example, a conventional dog “food” can be nutritionally complete and can provide the full range of the dog's daily nutrition requirements. Dog “food” can also be intended as the major source of the dog's calorific intake. In contrast, a chew need not provide such nutrition or calorific content.

A chew can further be distinguished from a “food” with regard to its size. The largest pieces in a food product are generally smaller than the size of a chew. For instance, PCT Appln. Pub NO. WO01/050882A discloses a food product which is reported as having a large size compared to other dried pet food, and discloses several examples. The largest of these examples is a triangular kibble having the following dimensions: thickness 16 mm, base 28 mm and sides 32 mm. In contrast, a chew can be relatively larger.

The oral chew of the present disclosure can be an individual piece having a largest dimension of at least about 40 mm, at least about 50 mm, at least about 60 mm, or at least about 70 mm. The size of the oral chew can vary in size according to the size of dog to which it is to be given. For example, chews for toy and small breeds can be smaller than chews for medium, large and giant breeds. Chews for larger breeds, for example, can be more than about 100 mm or more than about 120 mm in length.

A chew can be further distinguished from a “food” with regard to the time taken to consume a piece of chew compared to a piece of food. Normally, the consumption time for a piece of chew is longer than a piece of food. A piece of food can generally be consumed in less than about 10 seconds by an average sized dog, whereas a chew would take at least about 20 seconds for an average-sized dog to consume. In one embodiment, a chew of the present disclosure can take at least about 90 seconds or at least about 120 seconds for an average-sized dog to consume. In certain aspects, the chew of the present disclosure can exhibit a lasting time (in seconds) per gram of chew of at least about 3 seconds per gram of chew.

The oral chews of the present disclosure can include one or more starches. A person skilled in the art would appreciate a wide variety of starches are suitable for use in the present disclosure. The starch content of the chew of the present disclosure can be from about 10 wt % to about 80 wt % or from about 40 wt % to about 70 wt % relative to the total weight of the chew. As used herein, weights relative to the total weight of the chew are with respect to the finished product. In certain embodiments, the starch content of the chew can be from about 10 wt % to about 70 wt % to about 15 wt % to about 60 wt %, about 25 wt % to about 50 wt %, about 45 wt % to about 65 wt % to about 45 wt % to about 70 wt %, about 50 wt % to about 65 wt %, or about 50 wt % to about 60 wt % relative to the total weight of the chew. In certain embodiments, the chew can include at least about 10 wt %, at least about 15 wt %, about least about 25 wt %, at least about 40 wt %, at least about 45 wt %, at least about 50 wt %, at least about 55 wt %, at least about 60 wt %, at least about 65 wt %, or at least about 70 wt % starch, relative to the total weight of the chew. In particular embodiments, the chew can include about 40 wt %, about 45 wt %, about 50 wt %, about 55 wt %, about 60 wt %, about 65 wt %, or about 70 wt % starch, relative to the total weight of the chew. The relatively high starch content of the chew of the present disclosure can contribute to the chew's ability to retain its shape.

The oral chews of the present disclosure can include one or more humectants. A person skilled in the art would appreciate a wide variety of humectants are suitable for use in the present disclosure. The humectant content of the chew of the present disclosure can be from about 5 wt % to about 20 wt %, from about 10 wt % to about 20 wt %, from about 15 wt % to about 20 wt %, from about 5 wt %, to about 15 wt %, or from about 5 wt % to about 10 wt % relative to the total weight of the chew. In certain embodiments, the chew can include at least about 5 wt %, at least about 10 wt %, at least about 15 wt %, or at least about 20 wt % humectant, relative to the total weight of the chew. In particular embodiments, the chew can include about 5 wt %, about 10 wt %, about 15 wt %, or about 20 wt % humectant, relative to the total weight of the chew. The humectant content confers an ability of the chew to retain water.

The oral chews of the present disclosure can have a certain density. In particular embodiments, the chews can have a relatively low density. In certain embodiments, the chew can have a density of about 1.0 g cm⁻³ or less, about 0.9 g cm⁻³ or less, about 0.7 g cm⁻³ or less, about 0.6 cm⁻³, or about 0.5 g cm⁻³ or less. A person skilled in the art would appreciate that oral chews having various densities are suitable for use with the present disclosure.

In certain embodiments, the oral chews of the present disclosure can include one or more starches and one or more humectants. In particular embodiments, the oral chew can have a starch content of from about 10 wt % to about 80 wt % starch and about 5 wt %, c to about 20 wt % humectant, relative to the total weight of the chew. The chew can have a density of about 1.0 g cm⁻³ or less.

Oral chews of the present disclosure can have a texture that, when consumed by an animal such as a dog, contributes to maintaining the animal's oral health. The chew can exhibit a characteristic spongy texture, for example, due to its composition including the humectant content, which confers the ability to retain water, and a relatively low density. This texture allows the chew of the present disclosure to elastically rebound to a certain extent and so to at least partially close up a hole formed by an animal's tooth after the tooth is withdrawn during the chewing process. Thus, the chew of the present disclosure demonstrates an ability to self-heal and regain its shape to a certain extent.

In certain embodiments, oral chews of the present disclosure can be from about 2 kg to about 25 kg, about 2 kg to about 7 kg, about 5 kg to about 10 kg, about 7 kg to about 11 kg, about 10 kg to about 25 kg, about 11 kg to about 22 kg, or at least about 25 kg. In particular embodiments, the oral chew can be at least about 2 kg, at least about 5 kg, at least about 7 kg, at least about 10 kg, at least about 11 kg, at least about 15 kg, at least about 20 kg, at least about 22 kg, or at least about 25 kg. In certain embodiments, the oral chew can be about 2 kg, about 5 kg, about 7 kg, about 8 kg, about 10 kg, about 11 kg, about 12 kg, about 15 kg, about 18 kg, about 20 kg, about 22 kg, about 25 kg, about 28 kg, or about 30 kg. A person skilled in the art would appreciate that various sizes of oral chews are appropriate for use with the present disclosure.

### E. Methods of Use or Treatment

The methods of the disclosure subject matter can include administration or feeding the oral chew to the animal in order to modulate the oral microbiota. The methods of the disclosed subject matter are particularly well suited for use with companion animals, such as dogs, cats, and other domesticated animals.

In certain embodiments, the oral chew can be administered to the animal according to the weight of the animal, e.g., a dog. In certain embodiments, the dog can be of a toy, small, medium, large or giant breed. For example, and not by way of limitation, a toy dog breed can be administered an oral chew having a size in the range of from about 2 kg to about 7 kg; a small dog breed can be administered an oral chew having a size in the range of from about 7 kg to about 11 kg or from about 5 kg to about 10 kg; and a medium dog breed can be administered an oral chew having a size in the range of from about 10 kg to about 25 kg, about 10 kg to about 15 kg or from about 11 kg to about 22 kg. In certain embodiments, for example and not by way of limitation, large and giant dog breeds can be administered an oral chew having a size of at least about 25 kg.

In certain aspects, the oral chew can be administered to an animal with a clean mouth, e.g., after it has received a scale and polish, that has had its teeth cleaned, or that has generally healthy gingiva. Without being bound to a particular theory, the oral chew is thought to encourage the colonization of the clean mouth with more bacteria not associated with periodontal disease compared to a clean mouth to which an oral chew is not administered. In certain embodiments, the method can further include cleaning the animal's teeth or mouth.

In certain aspects, the oral chew can be administered daily, twice weekly, weekly or fortnightly. The chew can be fed to the dog for a period of 3 days, a year, for the life of the animal. In particular embodiments, the oral chew can be administered to the animal at least 5, 8, 10, 13, 15, 18, 20, 23, 25, 28, 35, 42 or more times.

The present disclosure provides kits that are useful in the modulation of the oral microbiota of an animal, e.g., to improve oral health of the animal. The kits can be used to administer and track administration of oral chews to an animal. In certain aspects, the kits can be used to collect a sample in which one or more bacteria associated with an oral disease or disorder (e.g., periodontal disease) or good oral health are detected. A kit for modulating the oral microbiota of an animal can generally include, amongst other things, sample collection devices for collection of samples, one or more oral chews for administering to the animal, and instructions with respect to appropriate diet regimens for the animal.

### EXAMPLES

For purpose of understanding and not limitation, the presently disclosed subject matter will be better understood by reference to the following Examples, which are provided as exemplary of the disclosed subject matter, and not by way of limitation.

### Example 1: Feeding Trials (Oral Care Chews)

The present example describes the effects of oral care chews included in a diet regimen in modulating the canine oral microbiota.

### Methods

Study Design

Twelve (12) Beagle dogs consisting of six (6) males (all neutered) and six (6) females (all intact) with an average age of 2.9 years (from 2.6 years to 3.4 years) and weighing from 10 kg to 25 kg participated in the study. The dogs were housed in pairs or groups in a temperature controlled indoor kennel overnight and during the day in outside paddocks enriched with shelter and toys. Water was available continuously.

A randomized cross-over study was undertaken in which one of two dietary regimes were fed: (1) commercially available main meal diet+oral care chew; and (2) commercially available main meal diet alone (control). Clinical observations were performed and the bacterial composition of canine dental plaque was assessed. The commercially available main meal diet comprised a 1:1 ratio of a complete and balanced commercial dry and commercial wet dog food. The test product was an extruded oral care chew (Pedigree® Dentastix®).

The study commenced with a 14-day pre-test phase followed by consecutive 28-day test phases. On day 1 of the pre-test phase, the dogs underwent general anesthesia for dental evaluations (gingivitis, plaque and calculus scores) and a full mouth scale and polish (S&P). For anesthesia, levomethadone hydrochloride (0.5 mg) and fenpipramid hydrochloride (0.025 mg) were administered for premedication and were followed by propofol (4 mg/kg body weight) to induce general anesthesia via an intravenous catheter. Anesthesia was maintained by inhalation with isoflurane delivered via an endotracheal tube. Prior to the D&P, supragingival plaque was collected from the buccal surfaces of all teeth using plastic microbiological loops (Scientific Laboratory Suppliers Ltd) (start pre-test phase). Following the D&P, the dogs received daily tooth brushing with water for the remainder of the pre-test phase. This was conducted after feeding of the main meal diet, approximately between 8 am to 9 am each morning with the aim of achieving clinically healthy gingiva prior to initiating the first test phase. Throughout the pre-test phase, all dogs received only the main meal diet, which was fed each morning between 7:30 am to 8 am during both the pre-test and test phases.

On day 1 of the first test phase, another buccal supragingival plaque sample was collected from all dogs under general anesthesia (end pre-test phase). They then received a full mouth D&P to provide clean tooth surfaces. During the test phase, dogs receiving chews were offered them daily approximately 5 hours after feeding of the main meal diet. Instances of refusal of either the main meal diet and/or chew were recorded. Approximately 24 hours after feeding of the last main meal diet or chew of the 28-day test phase, the dogs underwent general anesthesia during which supragingival plaque was collected (chew and no chew) and a D&P was conducted to prepare them for the next test phase.

The plaque samples were placed into 400 μl Tris-EDTA (TE) buffer (pH 8.0) in Eppendorf tubes and immediately placed on dry ice for up to 9 hours until the plaque samples had been collected from all the dogs. Samples were stored at −80° C. prior to DNA extraction and transported on dry ice during transit to a center for processing.

DNA Extraction

DNA was extracted from the plaque samples, as previously described by Davis et al. (2013), using the Epicentre Masterpure Gram Positive DNA Purification Kit (Epicentre, USA), according to the manufacturer's protocol with additional overnight lysis.

Amplification of 16S rDNA Gene

The variable V3-V4 regions of the 16S rDNA gene were amplified from the plaque DNA extractions. The universal bacterial primers to the 16S rDNA gene, 319F and 806R, each modified with a linker sequence, index sequence and heterogencity spacer as per (Fadrosh et al. 2014), were used for PCR amplification. The PCR mixtures (50 μl) contained 25 μl Phusion@ High-Fidelity PCR Master Mix with HF Buffer (MO531, New England Biolabs, UK), 5 μl of each primer (1 μM), 10 μl template DNA, 3.5 μl nuclease free water and 1.5 μl DMSO, prepared in a 96-well format. The PCR cycling conditions consisted of an initial denaturation step at 98° C. (30 s), followed by 30 cycles of 98° C. (15 s), 58° C. (15 s), and 72° C. (15 s), and a final elongation at 72° C. (60 s). Successful amplification was confirmed through electrophoresis of the PCR products on 1.5% agarose gels.

Library Preparations and Sequencing

Library preparation and sequencing were carried out. The 16S amplicons were pre-quantified using the Quant-iT™ PicoGreen@ dsDNA Assay Kit (Invitrogen, UK). The diluted amplicons were then quantified using the Fragment Analyzer (Advanced Analytical Technologies, Inc.), then pooled into groups of 121/122 samples. The library pools were gel-sized prior to sequencing on a MiSeq (Illumina) with v3 chemistry, 2x300 bp run modus.

Sequence Data Processing

Summary

Forward and reverse reads were assembled into a contiguous sequences spanning the entire V3-V4 regions using FLASH assembler (Magoc and Salzberg 2011). Tags were removed using TagCleaner (Schmieder et al. 2010) and sequences were demultiplexed in QIIME using split_libraries_fastq.py. Chimeric sequences were removed using userarch61(Edgar 2010). Sequences were clustered at >98% identity using uclust (Caporaso et al. 2010) to generate operational taxonomic units (OTUs) and the most abundant sequences were chosen as cluster representatives. These were annotated with blastall 2.2.25 (Altschul et al. 1990), which also contained canine and feline oral microbiome sequences previously published by the authors (Pruesse et al. 2007, Dewhirst et al. 2012, Dewhirst et al. 2015).

Details

A dual-indexing and assembly approach as described by Fadrosh et al., 2014 was used to sequence the V3-V4 region of the 16S gene on Illumina's MiSeq platform. Paired-end raw sequence data in FASTQ format was treated as follows: Sequences and 12-mer barcodes were separated using the trimfq function of seqtk (v 1.2-r94) (https://github.com/lh3/seqtk/blob/master/README.md). Forward and reverse reads were assembled using FLASH (v 1.2.10 XMagoč & Salzberg, 2011) with minimum and maximum overlaps of 40 and 200, respectively, and a maximum mismatch of 1% in the overlapping region. Tags were removed using TagCleaner (v 0.16)(Schmieder et al. 2010) with the settings −nomatch 3 −tag5 GGACTACHVGGGTWTCTAAT −mm5 3 −tag3 CTGCTGCCTCCCGTAGGAGT −mm3 3 (SEQ ID NO:1 and SEQ ID NO: 2, respectively). Sequences were split into samples using split_libraries_fastq.py (v 1.9.1) from QIIME (Caporaso et al., 2010) using a phred score cut-off of 30 (−q 29, representing 99.9% base call accuracy) and a barcode length of 24 (2×12). Chimeric sequences were removed using userarch6l with the setting “reference free” (Edgar, 2010). Sequences were then clustered using QIIME version 1.9.1 (ibid.), using pick_otus.py (v 1.9.1), which utilises uclust (v 1.2.22q)(Edgar, 2010) to cluster sequences with >98% identity (Caporaso et al., 2010) into Operational Taxonomic Units (OTUs). Uclust was run with modified parameters, with gap opening penalty set to 2.0 and gap extension penalty set to 1.0 and—A flag to ensure optimum alignment (Caporaso et al., 2010). For each OTU cluster the most abundant sequence was chosen as representative sequence using pick_rep_set.py (v 1.9.1) also from QIIME.

Relative abundance and distribution across samples was assessed for each OTU in order to separate noisy from consistent but rare OTUs (refer to Statistical Analysis section for detailed description). Representative sequences of all OTUs that passed the filtering criteria were searched against the Silva SSU database release 128 (Pruesse et al., 2007) using blastall (v2.2.25) (Altschul et al., 1990). If the alignment did not meet the cut-off criteria of ≥98% sequence identity and ≥98% query sequence coverage genus or higher level annotations were used.

Statistical Analysis

OTUs were combined in a single group of “rare” taxa if either they were present in each phase at an average proportion below 0.05% or were present in less than two samples in all phases. The 0.05% cut-off was selected based on statistical analysis of data from mock communities as referenced in Davis et al. 2013. To estimate and compare the relative abundance of OTUs, binomial generalised linear mixed models with a logit link were fit, using the count of an OTU out of the total number of sequences in a sample. Phase (start pre-test phase, end pre-test phase, chew and no chew) was included as the fixed effect and animal and an observation level random effect as the random structure (Harrison XA. (2014)). Mean proportions with 95% confidence intervals are reported for each phase and odds ratios with 95% confidence intervals are reported for all pairwise phase comparisons. All p-values were adjusted according to the false discovery method of (Benjamini and Hochberg 1995) to an overall test level of 5%. This analysis method was also applied for each phylum.

Multi-group principal component analysis (PCA) was performed on the log₁₀ proportions, with dog as the grouping variable, to determine if clustering of samples was apparent. Ellipses representing the 95% bivariate confidence region for PC1 and PC2 were calculated for each phase and included on the PCA score plot (Murdoch and Chow 2013).

Shannon diversity index was calculated for each sample and a linear mixed model was used to analyse the indices, with phase as the fixed effect and animal as the random effect. Means for each phase and differences between all pairs of phases are reported with 95% confidence intervals.

Statistical analyses were performed in R v3.3.3 (Team 2017) using lme4 (Bates et al. 2015), multcomp (Hothorn et al. 2008), ggplot2 (Wickham 2009), snowfall (Knaus 2013), mixOmics (Rohart et al. 2017), and ellipse libraries (Murdoch and Chow 2013).

Assigning Health Associations to OTUs

For the OTUs which showed a significant difference (p<0.05) and odds ratio >2 in the chew versus no chew comparison, health associations were assigned based on findings from previous studies (Davis et al. 2013, Wallis et al. 2015). Briefly, representative sequences of the OTUs from the previous studies (after removal of rare OTUs) were concatenated into a single FASTA file. Sequences were then clustered with QIIME. The representative V3-V4 sequence for each OTU in this study were queried against the representative sequences (V1-V3 sequence) from the previous studies using BLAST (Altschul et al. 2010). Based on a query match of ≥99% sequence identity the health status of the ‘matching’ OTU was inferred to the corresponding OTU from this study, thus enabling the information about bacterial associations with health and periodontal disease to be overlaid.

An OTU cluster was tagged with either health, health (weak), disease, disease (weak), none or unknown based on the known associations from the previous studies (ref: all 3 again). The term ‘weak’ was assigned if either the representative sequence could only be identified in one of the three studies or if the associations within an individual study were showing minor trends (odds ratio <2). An ‘unknown’ status was applied if the bacterial associations from the three studies did not form a consensus or the information from individual studies was not conclusive.

### Results

Samples and Sequence Quality

Supragingival plaque was sampled on four occasions from each of the twelve (12) Beagle dogs: i. a start pre-test phase; ii. end pre-test phase; iii. end test phase chew; and iv. end test phase no chew. The study generated a total of 47 supragingival plaque samples. A baseline start pre-test phase was not obtained for one (1) dog.

Illumina sequencing analysis of the 3′ end of the V3-V4 region of the 16S rDNA gene of the 47 supragingival plaque samples generated 6,946,238 assembled reads after processing through the bioinformatics pipeline. Final numbers of sequence reads per sample ranged from 55,666 to 302,682 with a median of 143,299 reads.

Bacterial Composition of Canine Supragingival Plaque

The 6,946,238 assembled sequences were assigned to 158 OTUs after filtering of “rare” sequence reads into a separate group as described in the methods. The rare group accounted for 2.37% of the total sequence.

Taxonomic assignment of each of the 158 OTUs resulted in 148 (93.7%) with ≥98% sequence identity to 16S sequences within the Silva database (v128). The remaining 10 OTUs aligned to sequences with between 92.1% and 97.9% identity. Of the 158 OTUs, 82 (51.9%) aligned to sequences previously identified as canine oral taxa (COTs) (Dewhirst et al. 2012) and 10 (6.3%) aligned to sequences previously identified as feline oral taxa (FOTs) (Dewhirst et al. 2015). The remaining 66 OTUs (41.8%) aligned to other taxa within the Silva database. Of these, 16 (10.1%) were designated species level taxonomy.

Analysis of the taxonomic composition of the 158 OTUs revealed that 141 belonged to nine (9) phyla: Proteobacteria (40.4%), Firmicutes (21.1%), Bacteroidetes (19.9%), Actinobacteria (7.5%), Synergistetes (1.4%), Spirochaetes (0.4%), Fusobacteria (0.3%), Tenericutes (0.06%) and Chlorobi (0.06%). The remaining 17 OTUs belonged to four (4) candidate phyla: Saccharibacteria (3.5%), Absconditabacteria (1.8%), Gracilibacteria (1.1%) and WS6 (0.08%).

The 25 most abundant taxa, present at ≥1%, accounted for approximately 74% of the sequences reads (Table 1). A novel Escherichia-Shigella species (OTU #6813) was the most abundant taxa representing 28.7% of the total number of sequence reads. A novel Bergeyella species (OTU #8463), Porphyromonas cangingivalis (OTU #5307) and a novel Proteocatella species (OTU #8440) were the next most abundant representing 7.13%, 6.27% and 2.37% of the sequence reads respectively. A novel Actinomyces species (OTU #2136) and Brachymonas sp. COT-015 (OTU #2778) represented 2.32% and 2.03% of the population respectively. A further 19 OTUs represented between 1.00% and 2.00% of the population and the remaining 133 OTUs were below 1.00% and ranged in relative abundance from 0.014% to 0.92%.

TABLE 1
The 25 most abundant operational taxonomic units (OTUs) present at >1% of total sequence reads in
supragingival plaque.

| | Assigned Taxonomy | Percentage | Total Number of | Percentage of total |
|---|---|---|---|---|
| OTU | (Family/Genus/Species) | identity | Sequence Reads | sequence reads (%) |
| 6813 | unclassified Escherichia-Shigella | 100.0 | 1992898 | 28.7 |
| | [novel] | | | |
| 8463 | unclassified Bergeyella [novel] | 100.0 | 495257 | 7.13 |
| 5307 | Porphyromonas cangingivalis | 100.0 | 435251 | 6.27 |
| 8440 | unclassified Proteocatella | 100.0 | 164446 | 2.37 |
| | [novel] | | | |
| 2136 | unclassified Actinomyces [novel 1] | 100.0 | 161439 | 2.32 |
| 2778 | Brachymonas sp. COT-015 | 99.07 | 140702 | 2.03 |
| 5126 | Peptostreptococcaceae bacterium | 100.0 | 133006 | 1.91 |
| | COT-005 | | | |
| 2726 | Filifactor villosus | 100.0 | 116581 | 1.68 |
| 9507 | TM7 phylum sp. COT-305 | 100.0 | 114527 | 1.65 |
| 9649 | unclassified Actinomyces [novel 6] | 100.0 | 113498 | 1.63 |
| 9738 | unclassified TM7/Saccharibacteria | 100.0 | 101225 | 1.46 |
| | [novel 4] | | | |
| 3427 | Granulicatella sp. COT-095 | 100.0 | 100406 | 1.45 |
| 10008 | Synergistales bacterium COT-178 | 100.0 | 95165 | 1.37 |
| 4982 | Lachnospiraceae bacterium | 100.0 | 91966 | 1.32 |
| | COT-026 | | | |
| 10386 | Lautropia sp. COT-175 | 100.0 | 91885 | 1.32 |
| 550 | Desulfovibrio sp. COT-070 | 99.77 | 87783 | 1.26 |
| 1536 | Peptostreptococcaceae bacterium | 100.0 | 87241 | 1.26 |
| | COT-047 | | | |
| 1724 | Porphyromonas gulae | 100.0 | 86822 | 1.25 |
| 3997 | unclassified Porphyromonas | 100.0 | 85555 | 1.24 |
| | [novel 2] | | | |
| 4559 | Peptostreptococcaceae bacterium | 100.0 | 84695 | 1.22 |
| | COT-135 | | | |
| 317 | Porphyromonas canoris | 100.0 | 77381 | 1.11 |
| 9348 | unclassified Actinomyces [novel 5] | 100.0 | 74720 | 1.08 |
| 7944 | unclassified SR1/Absconditabacteria | 99.75 | 73731 | 1.06 |
| | [novel 1] | | | |
| 7830 | Peptococcus sp. COT-044 | 100.0 | 69983 | 1.01 |
| 9842 | unclassified Neisseria [novel] | 100.0 | 69637 | 1.00 |

Comparison of Plaque Sample Groups between Phases

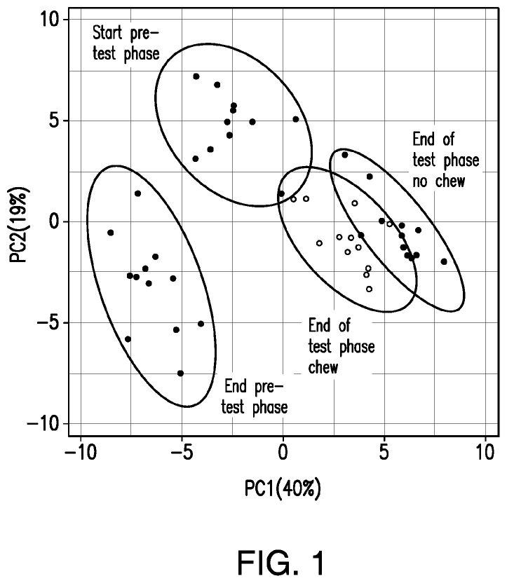

Principal component analysis (PCA) was used to visualize relatedness between phases. The first component explained 40% and the second component 19% of the variability in the OTU log₁₀ proportions (FIG. 1). Distinct clustering was observed between the four groups. The two test group dietary regimes shared the most commonality in OTU composition, shown in FIG. 1 by the proximity of the test phase samples.

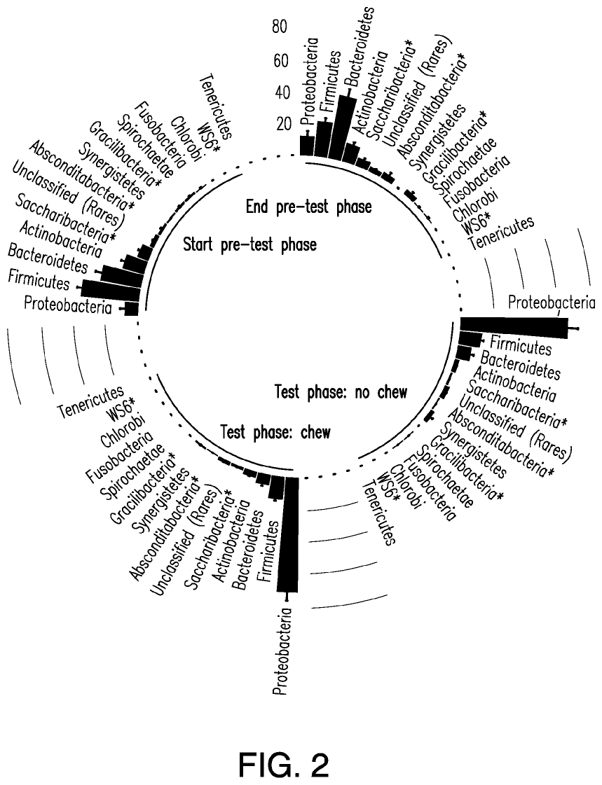

Across the timeframe of the study, the supragingival plaque sample groups displayed gross differences at the phylum level (FIG. 2). Phylum level composition was similar for the two test phase dietary regime groups where Proteobacteria was the most abundant phyla and both were found to be present at significantly higher proportions than the pre-test phase samples (p<0.01). In contrast, Bacteroidetes was the most abundant phyla in baseline sample at end of pre-test phase whereas Firmicutes was the most abundant phyla in baseline sample at start of pre-test phase. These proportions were found to be significantly higher than the corresponding proportions in each of the test phase groups (p<0.05).

For each OTU, pairwise comparisons were performed between the phase groups and revealed a similar number of OTUs to be significantly different between the comparisons ranging from 22 to 102 (Table 2). Sample groups for the test phase dietary regimes (no chew vs. chew) indicated the fewest number of differences across all the comparisons. A schematic to illustrate the taxonomy and changes in abundance for the significant OTUs for the no chew and chew comparison is shown in FIG. 3. The health, disease or lack of association for each taxa, based on knowledge from previous studies (see Methods), has also been indicated in FIG. 3. In summary, the chew intervention resulted in a significant increase in 6 health associated and 3 disease associated taxa compared to no chew (listed in Table 3). In contrast, 8 disease associated and 1 health associated taxa significantly increased in abundance with no chew compared to the chew group (Table 3). Across these comparisons, 1 taxa was found to have no health/disease association, while the health/disease association for 3 other taxa were unknown. Table 4 provides the phylum, class, order, family, genus and final taxomony classification of the OTUs.

TABLE 2
Operational taxonomic unit (OTU) summary comparing the
supragingival plaque groups. Indicates the number of taxa that
significantly differed between phase groups.

| | p-values < 0.05 |
|---|---|
| Start pre-test phase/End pre-test phase | 77 |
| Start pre-test phase/Test phase: no chew | 96 |
| Start pre-test phase/Test phase: chew | 84 |
| End pre-test phase/Test phase: no chew | 102 |
| End pre-test phase/Test phase: chew | 90 |
| Test phase: no chew/Test phase: chew | 22 |

TABLE 3

| | Taxonomy and changes in abundance for the significant | | | | | |
|---|---|---|---|---|---|---|
| | OTUs for the no chew and chew comparison. | | | | | |
| Test | Assigned | | | | | 95% |
| Phase | Health | | | Assigned Taxonomy | Fold | Confidence |
| Group | Status | | OTU | (Family/Genus/Species) | Change | Intervals |
| Chew | Health | 6 | 359 | unclassified Klebsiella | 1.16 | 1.15, 1.17 |
| | (weak) | | | [novel] | | |
| | | | 1400 | Propionibacterium sp. | 5.7 | 1.82, 17.9 |
| | | | | COT-296 | | |
| | | | 3753 | Catonella sp. COT-257 | 2.36 | 1.12, 4.98 |
| | | | 8491 | Corynebacterium mustelae | 3.83 | 1.64, 8.97 |
| | | | 8712 | Prevotella sp. COT-282 | 2.52 | 1.23, 5.18 |
| | | | 9507 | TM7 sp. COT-305 | 2 | 1.98, 2.01 |
| | Disease | 1 | 6186 | Parvimonas sp. COT-101 | 5.75 | 1.34, 24.6 |
| | Disease | 2 | 6 | unclassified Actinomyces | 4.24 | 1.97, 9.1 |
| | (weak) | | | [novel 3] | | |
| | | | 2829 | unclassified Treponema | 1.06 | 1.06, 1.07 |
| | | | | [novel 2] | | |
| No | Health | 1 | 550 | Desulfovibrio sp. COT-070 | 6.59 | 1.72, 25.3 |
| Chew | (weak) | | | | | |
| | Disease | 3 | 6994 | Fretibacterium sp. FOT- | 4.73 | 1.89, 11.9 |
| | | | | 218 | | |
| | | | 7484 | Helcococcus sp. FOT_023 | 2.93 | 2.9, 2.95 |
| | | | 10946 | unclassified Clostridium | 3.61 | 3.58, 3.64 |
| | | | | [novel] | | |
| | Disease | 5 | 517 | Desulfomicrobium orale | 1.71 | 1.7, 1.73 |
| | (weak) | | | | | |
| | | | 1640 | Anaerovorax sp. COT-125 | 3.51 | 1.26, 9.75 |
| | | | 1883 | Bacterodia bacterium | 3.93 | 1.48, 10.5 |
| | | | | COT-387 | | |
| | | | 9916 | Neisseria canis | 5.56 | 1.45, 21.3 |
| | | | 9723 | Pelistega sp. COT-267 | 12.8 | 3.37, 48.5 |

TABLE 4
OTU classification

| OTU | Phylum | Class | Order | Family | Genus | Final Taxonomy |
|---|---|---|---|---|---|---|
| 359 | Proteobacteria | Gammaproteobacteria | Enterobacteriales | Enterobacteriaceae | Klebsiella | unclassified |
| | | | | | | Klebsiella [novel] |
| 1400 | Actinobacteria | Actinobacteria | Propionibacteriales | Propionibacteriaceae | Propionibacterium | Propionibacterium |
| | | | | | | sp. COT-296 |
| 3753 | Firmicutes | Clostridia | Clostridiales | Lachnospiraceae | Catonella | Catonella sp. |
| | | | | | | COT-257 |
| 8491 | Actinobacteria | Actinobacteria | Corynebacteriales | Corynebacteriaceae | Corynebacterium | Corynebacterium- |
| | | | | | | mustelae |
| 8712 | Bacteroidetes | Bacteroidia | Bacteroidales | Prevotellaceae | Prevotella | Prevotella sp. |
| | | | | | | COT-282 |
| 9507 | Saccharibacteria | — | — | — | — | TM7 sp. COT- |
| | (TM7) | | | | | 305 |
| 6186 | Firmicutes | Tissierellia | Tissierelliales | Peptoniphilaceae | Parvimonas | Parvimonas sp. |
| | | | | | | COT-101 |
| 6 | Actinobacteria | Actinobacteria | Actinomycetales | Actinomycetaceae | Actinomyces | Actinomyces |
| | | | | | | unclassified |
| | | | | | | [novel 3] |
| | | | | | | unclassified |
| 2829 | Spirochaetaes | Spirochaetia | Spirochaetales | Spirochaetaceae | Treponema | Treponema |
| | | | | | | [novel 2] |
| 550 | Proteobacteria | Deltaproteobacteria | Desulfovibrionales | Desulfovibrionaceae | Desulfovibrio | Desulfovibrio sp. |
| | | | | | | COT-070 |
| 6994 | Synergistetes | Synergistia | Synergistales | Synergistaceae | Fretibacterium | Fretibacterium |
| | | | | | | sp. FOT-218 |
| 7484 | Firmicutes | Clostridia | Clostridiales | Peptostreptococcaceae | Helcococcus | Helcococcus sp. |
| | | | | | | FOT-023 |
| | | | | | | unclassified |
| 10946 | Firmicutes | Clostridia | Clostridiales | — | — | Clostridium |
| | | | | | | [novel] |
| 517 | Proteobacteria | Deltaproteobacteria | Desulfovibrionales | Desulfomicrobiaceae | Desulfomicrobium | Desulfomicrobium |
| | | | | | | orale |
| 1640 | Firmicutes | Clostridia | Clostridiales | Eubacteriaceae | Anaerovorax | Anaerovorax sp. |
| | | | | | | COT-125 |
| | | | | | | Bacterodia |
| 1883 | Bacteroidetes | Bacteroidia | — | — | — | bacterium COT- |
| | | | | | | 387 |
| 9916 | Proteobacteria | Betaproteobacteria | Neisseriales | Neisseriaceae | Neisseria | Neisseria canis |
| 9723 | Proteobacteria | Betaproteobacteria | Burkholderiales | Alcaligenaceae | Pelistega | Pelistega sp. |
| | | | | | | COT-267 |

Diversity

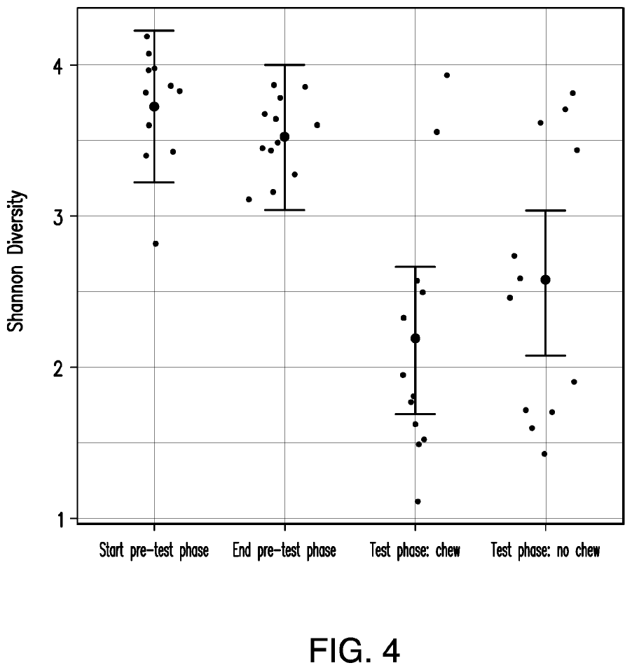

The linear mixed model used to analyse the Shannon diversity index data showed some significant differences between the groups of plaque samples (FIG. 4). The Shannon diversity index was significantly lower for the test phase samples compared to the pre-test phase samples (p<0.05). Index values for the two test phase samples (chew and no chew) and for both pre-test phase samples were not significantly different (p>0.05). OTU sequences are provided in Table

TABLE 5
OTU Sequences

| Sequence | |
|---|---|
| identifier | 16S OTU Sequence |
| 359 | CCTGTTTGCTCCCCACGCTTTCGCACCTGA |
| | GCGTCAGTCTTTGTCCAGGGGGCCGCCTTC |
| | GCCACCGGTATTCCTCCAGATCTCTACGCA |
| | TTTCACCGCTACACCTGGAATTCTACCCCC |
| | CTCTACAAGACTCTAGCCTGCCAGTTTCGA |
| | ATGCAGTTCCCAGGTTGAGCCCGGGGATTT |
| | CACATCCGACTTGACAGACCGCCTGCGTGC |
| | GCTTTACGCCCAGTAATTCCGATTAACGCT |
| | TGCACCCTCCGTATTACCGCGGCTGCTGGC |
| | ACGGAGTTAGCCGGTGCTTCTTCTGCGGGT |
| | AACGTCAATCGACAAGGTTATTAACCTTAT |
| | CGCCTTCCTCCCCGCTGAAAGTACTTTACA |
| | ACCCGAAGGCCTTCTTCATACACGCGGCAT |
| | GGCTGCATCAGGCTTGCGCCCATTGTGCAA |
| | TATTCCCCA |
| | [SEQ ID NO. 3] |
| 1400 | GCCTGTTCGCTCCCCACGCTTTCGCTTCTC |
| | AGCGTCAGGAAAGGTCCAGAGAACCGCCTT |
| | CGCCACCGGTGTTCCTCCTGATATCTGCGC |
| | ATTCCACCGCTCCACCAGGAATTCCGTTCT |
| | CCCCTACCTCCCTCAAGTCAGCCCGTATCG |
| | AAAGCAAGCTCAGAGTTAAGCCCTGAGTTT |
| | TCACTCCCGACGCGACAAACCGCCTACAAG |
| | CTCTTTACGCCCAATAAATCCGGACAACGC |
| | TCGCACCCTACGTATCACCGCGGCTGCTGG |
| | CACGTAGTTAGCCGGTGCTTCTTCTGTCGG |
| | TACCGTCACTCACGCTTCGTCCCGACTGAA |
| | AGCGGTTTACAACCCGAAGGCCGTCATCCC |
| | GCACGCGGCGTTGCTGCATCAGGCTTCCGC |
| | CCATTGTGCAATATTCCCCA |
| | [SEQ ID NO. 4] |
| 3753 | CCTGTTTGCTACCCACGCTTTCGAGCCTCA |
| | GCGTCAGTTTTGGTCCAGCAAGCCGCCTTC |
| | GCCACCGGTGTTCTTCCTAATATCTAAGCA |
| | TTTCACCGCTACACTAGGAATTCCGCTTGC |
| | CTCTCCCATACTCAAGCCTAACAGTTTTGG |
| | GAGCAGTCTCGGGGTTGAGCCCCGAGCTTC |
| | CACTCTCAACTTGAAAGGCCGCCTGCGCTC |
| | CCTTTACACCCAGTAAATCCGGATAACGCT |
| | TGCCCCCTACGTATTACCGCGGCTGCTGGC |
| | ACGTAGTTAGCCGGGGCTTCTTAGTCAGGT |
| | ACCGTCATCATCTTCCCTGCTGATAGAGCT |
| | TTACATACCGAAATACTTCTTCACTCACGC |
| | GGCGTCGCTGCATCAGAGTTTCCTCCATTG |
| | TGCAATATCCCCCA |
| | [SEQ ID NO. 5] |
| 8491 | CCTGTTCGCTCCCCATGCTTTCGCTCCTCA |
| | GCGTCAGTTACTGCCCAGAGACCTGCCTTC |
| | GCCATCGGTGTTCCTCCTGATATCTGCGCA |
| | TTCCACCGCTACACCAGGAATTCCAGTCTC |
| | CCCTACAGCACTCCAGTTATGCCCGTATCG |
| | CCTGCAACCCCGAAGTTAAGCCCCGGTATT |
| | TCACAGACGACGCAACAAACCACCTACGAG |
| | CTCTTTACGCCCAGTAATTCCGGACAACGC |
| | TCGCACCCTACGTATTACCGCGGCTGCTGG |
| | CACGTAGTTAGCCGGTGCTTCTTCTACAGG |
| | TACCGTCACCTTAAAAAAGGCTTCGTCCCT |
| | ACCGAAAGAGGTTTACAACCCGAAGGCCGT |
| | CATCCCCCACGCGGCGTCGCTGCATCAGGC |
| | TTGCGCCCATTGTGCAATATTCCCCA |
| | [SEQ ID NO. 6] |
| 8712 | CCTGTTTGATACCCACACTTTCGAGCCTCA |
| | GCGTCAGTTGTGCTCCCGGCATATGCCTTC |
| | GCGATCGGAGTTCTTCGTAATATCTAAGCA |
| | TTTCACCGCTACACTACGAATTCCAATGCC |
| | GCTGCGCACACTCAAGACAACCAGTATCAA |
| | CTGCAATTTTAAGGTTGAGCCTCAAACTTT |
| | CACAGCTGACTTAATCATCCGCCTACGCTC |
| | CCTTTAAACCCAATAAATCCGGATAACGCC |
| | CGAACCTTCCGTATTACCGCGGCTGCTGGC |
| | ACGGAATTAGCCGGTCCTTTTTCTTACGGT |
| | ACTTGCAAGACACCACACGTGGCGTTTTTT |
| | ACCCCCGTATAAAAGCAGTTTACAACCCAG |
| | AGGGCAGTCTTCCTGCACGCTACTTGGCTG |
| | GTTCAGACTCTCGTCCATTGACCAATATTC |
| | CTCA |
| | [SEQ ID NO. 7] |
| 9507 | CCCGTTCGCTCCCCACGCTTTCGTGCCTTA |
| | GCGTCAGAAATGGTCCAGTAACCTGCCTAC |
| | GCCATTGGTGTTCCTTCTAATATCTACGGA |
| | TTTCACTCCTACACTAGAAATTCCAGTTAC |
| | CTCTACCACTCTCGAGTTTAGCAGTTTGAA |
| | TAATAGTCTGTATGGTTGAGCCACCAGGTT |
| | TCACTATTCACTTACTAAACCGCCTACGCA |
| | ACTCTTTACGCCCAGTCACTCCGGATAATG |
| | CTTGCACCCTACGTATGACCGCGGCTGCTG |
| | GCACGTAGTTAGCCGGTGCTTATTCATGAG |
| | TTACCGTCATATTCTTCACTCATAAAAGAA |
| | GTTTACAACCCGAAGGCCTTCATCCTTCAC |
| | GCGGCGTTGCTCCATCAGGCTTTCGCCCAT |
| | TGTGGAAGATTCCTCA |
| | [SEQ ID NO. 8] |
| 6186 | CCTGTTTGCTCCCCACGCTTTCGTACCTGA |
| | GCGTCAGTAAAAGTCCAGAAAGTCGCCTTC |
| | GCCACCGGTATTCCTCCTAATATCTACGCA |
| | TTTCACCGCTACACTAGGAATTCCACTTTC |
| | CTCTCCTTCACTCAAGCCTTCCAGTTTCAA |
| | GTGCTTAATGAGGTTAAGCCTCACGCTTTC |
| | ACACCTGACTTAAAAGGCCGCCTACGTACC |
| | CTTTACGCCCAATAATTCCGGACAACGCTC |
| | GCCCCATACGTATTACCGCGGCTGCTGGCA |
| | CGTATTTAGCCGGGGCTTCCTCCTATGATA |
| | CCGTCATTATCTTCTCATAGGACAGAGCTT |
| | TACGACTCGAAAGCCTTCTTCGCTCACGCG |
| | GCGTCGCTGCATCAGGGTTTCCCCCATTGT |
| | GCAATATTCCCCA |
| | [SEQ ID NO. 9] |
| 6 | CCTGTTCGCTCCCCACGCTTTCGCTCCTCA |
| | GCGTCAGTAACGGCCCAGAGACCCGCCTTC |
| | GCCACCGGTGTTCCTCCTGATATCTGCGCA |
| | TTCCACCGCTACACCAGGAATTCCAGTCTC |
| | CCCTACCGCACTCAAGCCAGCCCGTACCCA |
| | CCGCAAGCCCGGAGTTAAGCCCCGGGTTTT |
| | CACGGCAGACGCGACAAGCCGCCTACAAGC |
| | CCTTTACGCCCAATAATTCCGGACAACGCT |
| | CGCGCCCTACGTATTACCGCGGCTGCTGGC |
| | ACGTAGTTAGCCGGCGCTTCTTTACCCACT |
| | ACCCTCAACTAGAACAAAAACTAGCCTTGA |
| | CCATGAGCGAAAGAGGTTTACAACCCGAAG |
| | GCCGTCATCCCTCACGCGGCGTTGCTGCAT |
| | CAGGCTTGCGCCCATTGTGCAATATTCCCC |
| | A |
| | [SEQ ID NO. 10] |
| 2829 | CCTGTTTGCTCCCCGCACCTTCGCATATCA |
| | GCGTCAATCATCGGCCAGAAACCCGCCTTC |
| | GCCACCGGTGTTCTTCCAAATATCTACAGA |
| | TTCCACCCCTACACTTGGAATTCCGGTTTC |
| | CCCTCCGTGATTCAAGTTAAGCAGTACCCA |
| | ATGCAGTTTACGAGTTAAGCTCGTAGATTT |
| | CACATCAGGCTTACCTAACCGCCTACATGC |
| | CCTTTACGCCCAATAATTCCGAACAACGCT |
| | TGGGGCTTACGTGTTACCGCGGCTGCTGGC |
| | ACGTAATTAGCCGCCCCTTATTCGCATGAT |
| | TACCGTCATCAGATAGGCATTCCCTCCTAT |
| | CCTTATTCTTCATCTGCAAAAGAACTTTAC |
| | AACCTTTCGGCCTTCATCGTTCACGCGGCG |
| | TCGCTCCGTCAGACTTTCGTCCATTGCGGA |
| | AGATTCTTAG |
| | [SEQ ID NO. 11] |
| 6992 | CCCTTTCGCTCCCCACGCTTTCGTCTCTCA |
| | GTGTCAGTAGTGTTCCAGCAAGCTGCCTTC |
| | GCTTTTGGTATTCTGGTATGTATCAACGGA |
| | TTGCACCCCTACTCATACCGTTCTGCTTGC |
| | CTCTCCCACACTCAAGTTATATGGTTTTCC |
| | GCGCAATCCAGGGTTGAGCCCTGGAGTTTC |
| | ACACGAAACCTTTATAACCACCTACAGACG |
| | CTTTACGCCCAGTAATTCCGGATAACGCTT |
| | GGGGCCCTCGTATTACCGCGGCTGCTGGCA |
| | CGAAGTTTGCCGCCCCTTATTCCTCTCGTA |
| | CCGTCATTATCTTCCGAGAGAAAAGAAGTT |
| | TACACCAACAAAGGCTTCATCCTTCACGCG |
| | GTGTCGCTCCATCAGGCTTTCGCCCATTGT |
| | GGAAGATTCCTCA |
| | [SEQ ID NO. 12] |
| 550 | CCTGTTTGCTCCCCACGCTTTCGCACCTCA |
| | GCGTCAATACCGGTCCAGGTGGCCGCCTTC |
| | GCCACTGATGTTCCTCCAGATATCTACGGA |
| | TTTCACTCCTACACCTGGAATTCCGCCACC |
| | CTCTCCCGGATTCAAGTCACGCAGTATCAA |
| | AGGCAGTTCCACGGTTGAGCCGTGGGATTT |
| | CACCCCTGACTTACATGACAGCCTACGTGC |
| | GCTTTACGCCCAGTAATTCCGATTAACGCT |
| | CGCACCCTCCGTATTACCGCGGCTGCTGGC |
| | ACGGAGTTAGCCGGTGCTTCCTTTGAAGGT |
| | ACCGTCAATACACCCCTGATTGGCAGAGTG |
| | CACCTTCTTCCCTTCCGACAGAGGTTTACG |
| | ATCCGAAAACCTTCATCCCTCACGCGGCGT |
| | CGCTGCGTCAGGCTTTCGCCCATTGCGCAA |
| | TATTCCCCA |
| | [SEQ ID NO. 13] |
| 6994 | CCTGTTTGCTCCCCACGCTTTCGCACCTGAG |
| | CGTCAGTTACCGTCCAGCAAGTCGCCTTCG |
| | CCACCGATGTTCTTCCCAATATCTACGCAT |
| | TTCACCGCTACACTGGGAATTCCACTTGCC |
| | TCTCCGGTACTCCAGCACCTCAGTCTCAAC |
| | TGCATAACACGGTTAAGCCGCATCCTTTAA |
| | CAGCTGACTTGAAGCACAGCCTGCGTGCCC |
| | TTTACGCCCAGTAATTCCGGACAACGCTCG |
| | CCCCCTACGTATTACCGCGGCTGCTGGCAC |
| | GTAGTTAGCCGGGGCTTATTCATGTGGTAC |
| | CGTCACTCTCTTCTTCCCACATAAAAGAAC |
| | TTTACGACCCGAAGGCCTTCTTCGTTCACG |
| | CGGCGTCGCTGGGTCAGGATTCCTCCCATT |
| | GCCCAATATTCCCCA |
| | [SEQ ID NO. 14] |
| 7484 | CCTGTTTGCTCCCCACGCTTTCGTACCTCA |
| | GCGTCAGTTAGATTCCAGAAAGTCGCCTTC |
| | GCCACCGGTATTCCTCCAAATATCTACGCA |
| | TTTCACCGCTACACTTGGAATTCCACTTTC |
| | CCCTCATCTACTCAAGTTATCCAGTTTCCA |
| | CACCTTACATTGGTTGAGCCAATGCCTTTT |
| | AATATGGACTTAAATAACCGCCTACGTACC |
| | CTTTACGCCCAATAATTCCGGACAACGCTC |
| | GCCCCATACGTATTACCGCGGCTGCTGGCA |
| | CGTATTTAGCCGGGGCTTTCTTCTTGGTTA |
| | CTGTCATTATCTTCACCAAGGACAGAACTT |
| | TACAACCCGAAGGCCTTCTTCGTTCACGCG |
| | GCGTCGCTGCATCAGGGTTTCCCCCATTGT |
| | GCAAAATTCCCCA |
| | [SEQ ID NO. 15] |
| 10946 | CCTGTTTGCTCCCCACGCTTTCGAGCCTCA |
| | GCGTCAGTTACAGTCCAGAGAGTCGCCTTC |
| | GCCACTGGTGTTCTTCCTAATCTCTACGCA |
| | TTTCACCGCTACACTAGGAATTCCACTCTC |
| | CTCTCCTGCACTCTAGATAACCAGTTTGGA |
| | ATGCAGCACCCAAGTTGAGCCCGGGTATTT |
| | CACATCCCACTTAATCATCCGCCTACGCTC |
| | CCTTTACGCCCAGTAAATCCGGATAACGCT |
| | CGCCACCTACGTATTACCGCGGCTGCTGGC |
| | ACGTAGTTAGCCGTGGCTTCCTCCTTGGGT |
| | ACCGTCATTATCTTCCCCAAAGACAGAGCT |
| | TTACGATCCGAAAACCTTCATCACTCACGC |
| | GGCGTTGCTGCATCAGGGTTTCCCCCATTG |
| | TGCAATATTCCCCA |
| | [SEQ ID NO. 16] |
| 517 | CCTGTTTGCTCCCCACACTTTCGCACCTCA |
| | GCGTCAATACCTGTCCAGGCGGCCGCCTTC |
| | GCCACCGGTGTTCCTCCTGATATCTACGGA |
| | TTTCACTCCTACACCAGGAATTCCGCCGCC |
| | CTCTCCAGGATTCGAGCCCCGCAGTTTCAA |
| | GTGCAGTTCCACGGTTGAGCCGTGGGATTT |
| | CACACCTGACTTACAAGGCCGCCTACGTGC |
| | GCTTTACGCCCAGTAATTCCGAATAACGCT |
| | TGCACCCTCCGTATTACCGCGGCTGCTGGC |
| | ACGGAGTTAGCCGGTGCTTCCTTTGAAGGT |
| | ACCGTCAAAATGCGGGCCTATTGGACCCGC |
| | ATCACTTCTTCCCTTCTGACAGAGGTTTAC |
| | GATCCGAAAACCTTCATCCCTCACACGGCG |
| | TTGCTGCGTCAGGCTTTCGCCCATTGCGCA |
| | ATATTCCCCA |
| | [SEQ ID NO. 17] |
| 1640 | CCTGTTTGCTACCCACGCTTTCGTGCCTCA |
| | GTGTCAGTTACAGTCCAGAAAGCCGCCTTC |
| | GCCACCGGTGTTCCTCCTAATATCTACGCA |
| | TTTCACCGCTACACTAGGAATTCCACTTTC |
| | CCCTCCTGCACTCAAGCTACACAGTTCGCA |
| | GGGCTTACAATGGTTAAGCCACTGCCTTTC |
| | ACCCCACGCTTATCTAGCCACCTACGCACT |
| | CTTTACGCCCAATAATTCCGGATAACGCTC |
| | GCCCCCTACGTATTACCGCGGCTGCTGGCA |
| | CGTAGTTAGCCGGGGCTTTCTTGATAGGTA |
| | CCGTCACCTTTTTCTTCCCTATCGACAGAG |
| | CTTTACGACCCAAAGGCCTTCTTCGCTCAC |
| | GCGGCGTTGCTGCATCAGGCTTTCGCCCAT |
| | TGTGCAATATTCCCCA |
| | [SEQ ID NO. 18] |
| 1883 | CCTGTTCGATACCCACGCCTTCGTGCATCA |
| | GCGTCAATGAGGGGCTCGCGAGATGCCTTC |
| | GCAATCGGTGTTCTGTGTGATATCTATGCA |
| | TTTCACCGCTACACCACACATTCCTCCCGC |
| | GGCGCCCCAATTCAAGCGCGACAGTTTCGA |
| | CGGCAAACCGCGCGTTGAGCGCGAGGATTT |
| | CACCGCCGACTTGACACGCAGCCTACGCAC |
| | CCTTTAAACCCAATAAATCCGGATAACGCT |
| | CGCATCCCCCGTATTACCGCGGCTGCTGGC |
| | ACGGAGTTAGCCGATGCTTATTCGACCGGT |
| | ACTCTCATCGGGCCACCAGTGGCCCTTATT |
| | GCTCCCGGTCAAAAGGAGTTTAAGACCCGT |
| | AGGGCCGTCGCCTCCACGCGGCATGGCTGG |
| | ATCAGGCTTACGCCCATTGTCCAATATCCC |
| | TCA |
| | [SEQ ID NO. 19] |
| 9916 | CCTGTTTGCTACCCACGCTTTCGAGCATGA |
| | ACGTCAGTATTATCCCAGGGGGCTGCCTTC |
| | GCCATCGGTATTCCTCCACATCTCTACGCA |
| | TTTCACTGCTACACGTGGAATTCTACCCCC |
| | CTCTGACATACTCTAGTTACCCAGTTCAGA |
| | ACGCCGTTCCCAGGTTAAGCCCGGGGATTT |
| | CACATCCTGCTTAAGTAACCGTCTGCGCTC |
| | GCTTTACGCCCAGTAATTCCGATTAACGCT |
| | CGCACCCTACGTATTACCGCGGCTGCTGGC |
| | ACGTAGTTAGCCGGTGCTTATTCTTACGGT |
| | ACCGTCATAACTTCAGGGTATTAGCCCAAA |
| | GCCTTTCTTCCCGTACAAAAGTCCTTTACA |
| | ACCCGAAGGCCTTCTTCAGACACGCGGCAT |
| | GGCTGGATCAGGGTTCCCCCCATTGTCCAA |
| | AATTCCCCA |
| | [SEQ ID NO. 20] |
| 9723 | CCTGTTTGCTCCCCACGCTTTCGTGCATGA |
| | GCGTCAGTATTATCCCAGGGGGCTGCCTTC |
| | GCCATCGGTATTCCTCCACATCTCTACGCA |
| | TTTCACTGCTACACGTGGAATTCTACCCCC |
| | CTCTGACATACTCTAGTTCGGGAGTTAAAA |
| | ATGCCGTTCCAAGGTTGAGCCCTGGGATTT |
| | CACATCTTTCTTTCCGAACCGCCTGCGCAC |
| | GCTTTACGCCCAGTAATTCCGATTAACGCT |
| | TGCACCCTACGTATTACCGCGGCTGCTGGC |
| | ACGTAGTTAGCCGGTGCTTATTCTTCAGGT |
| | ACCGTCATCACGCAAAGGTATTAACTCTGC |
| | GCTTTTCTTCCCTGACAAAAGTGCTTTACA |
| | ACCCGAAGGCCTTCATCGCACACGCGGGAT |
| | GGCTGGATCAGGGTTCCCCCCATTGTCCAA |
| | AATTCCCCA |
| | [SEQ ID NO. 21] |
| 1221 | CCTGTTTGCTCCCCACGCTTTCGCGCCTCA |
| | GCGTCAGTTAATGTCCAGCAGGCCGCCTTC |
| | GCCACTGGTGTTCCTCCCTATATCTACGCA |
| | TTTCACCGCTACACAGGGAATTCCGCCTGC |
| | CTCTCCATCACTCAAGAACTACAGTTTCAA |
| | GTGCACGCTCGGGGTTGAGCCCCGAGATTT |
| | CACACCTGACTTGCAGTCCCGCCTACACGC |
| | CCTTTACACCCAGTAAATCCGGATAACGCT |
| | TGCCACCTACGTATTACCGCGGCTGCTGGC |
| | ACGTAGTTAGCCGTGGCTTATTCCTGAAGT |
| | ACCGTCATTATCTTCCCTCAGAAAAGAAGT |
| | TTACAACCCGAAAGCCTTCTTCCTTCACGC |
| | GGCGTTGCTGGGTCAGGCTTGCGCCCATTG |
| | CCCAATATTCCCCA |
| | [SEQ ID NO. 22] |
| 10474 | CCGTTTTGCTACCCACGCTTTCGAGCCTTA |
| | GTGTCAGACAAGGACCAGCGAATCGCCTTC |
| | GCCACTGATGTTCCTCCCAATATCAACGCA |
| | TTTCACCGCTCCACTGGGAATTCCATTCGC |
| | CTCTTCCTGTCTCAATTCCTGTAGTTTCCA |
| | AAGCTTTCCCACGGTTGAGCCGTGGTCTTT |
| | AACTAAAGACTTACAGAAACACCTACGCAT |
| | CTCTATACACCCAATAAATCCGGATAACGT |
| | TCGCACCCTACGCATCACCGATGCTTCTGG |
| | CACGTAGTTAGCAGGTGCTTATTCATATGG |
| | TACCGTCATCTATTCTTCCCATATAAAAGG |
| | AGTTTACAATCCGAAGACCGTCATCCTCCA |
| | CACTGTGTCGCTGCGTCAGGGTTGCCCCCA |
| | TTGCGCAAGATTCCTAAT |
| | [SEQ ID NO. 23] |
| 412 | CCTGTTTGCTCCCCACGCTTTCGTGCCTCA |
| | GCGTCAGTATAGGCCCAGCAAGCCGCCTTC |
| | GCCACTGGTGTTCCTCCATATATTTACGCA |
| | TTTTACCGCTACACATGGAATTCCACTTGC |
| | CTCTACCTAACTCTAGTCTCCCAGTTTTCA |
| | AAGCGTTCCAAAGTTGAGCTTTGGATTTAA |
| | ACCTTGAACTTAAAAAACCGCCTACGCACC |
| | CTTTACGCCCAATAATTCCGGATAACGCTT |
| | GCCCCCTATGTATTACCGCGGCTGCTGGCA |
| | CATAGTTAGCCGGGGCTTATTCATTTAGTA |
| | CCGTCAATATCATATCATTTCCTATACAAT |
| | ATGTTCTTCCTAAATAAAAGAATTTTACGT |
| | ACTAGAAGTATGTCTTCATTCACGCGGTAT |
| | CGCTCGGTCAGGGTTTCCCCCATTGCCGAA |
| | GATTCTCTA |
| | [SEQ ID NO. 24] |

### DISCUSSION

Investigations into the efficacy of canine dental chews have classically focused on clinical plaque and calculus indices (Gorrel and Bierer 1999, Gorrel et al. 1999, Brown and McGenity 2005, Hennet et al. 2006, Clarke et al. 2011, Quest 2013). The relationship of both of these factors to the development of periodontal disease is understood and a particular product's potential to reduce either or both supports prevention.

On a microbial front, the advent of high-throughput molecular analyses tools such as 16s rRNA sequencing have enabled research to define the canine plaque microbiota (Elliott et al. 2005, Dewhirst et al. 2012) and to investigate the associations of specific bacterial species to healthy gingiva or periodontal disease (Riggio et al. 2011, Davis et al. 2013, Wallis et al. 2015, Davis 2016). By consolidation of the information available to date, this study has advantageously shown that shifts in microbiota can also be used to demonstrate efficacy, illustrating that bacteria communities within plaque can be manipulated towards health within a short period of time by feeding a daily oral care chew. The present study is a first in demonstrating the effect of regular feeding of an oral care chew on the canine oral microbiota.

Considering the employment of a single breed within this study, extrapolation of these findings to breed sizes either side of medium dogs can be of concern. Small, teenie and toy breeds for example are thought to exhibit different chewing behaviors, particularly with regards to chews. Key insights regarding health and disease associated bacterial species to date stem from a study of pure and mixed breeds (Davis et al. 2013). This and other studies primarily focus on subgingival plaque microbial populations. Unpublished data from a similar study to the one conducted here has shown a positive impact on both subgingival and supragingival plaque microbiota upon feeding miniature schnauzers the appropriately sized equivalent chew. Not only does this demonstrate the transferability of the findings to other breeds, but supports the rationale to collect and analyze only supragingival plaque microbiota in the current study. Key representative health associated species have also been shown to reside within early supragingival plaque biofilms (Holcombe et al. 2014). Not only that, but the development of the biofilms and associated ability to be able to measure shifts in microbial populations between 24 and 48 hours post D&P also supports the use of supragingival plaque in microbial biomarker detection (Holcombe et al. 2014).

Taxonomic assignment of the OTUs identified in this study found just over half aligned to previously characterised COT and FOT sequences while 31.6% could not be discriminated at the species level and were given novel taxonomic identities. When compared to other studies profiling canine oral microbiota, these numbers for COT/FOT and novel sequences are far lower and higher, respectively (Davis et al. 2013, Wallis et al. 2015). The difference in these numbers can be a consequence of the collection of supragingival plaque, as an alternative to subgingival plaque. Although the belief is that the key most abundant species are reflected well in each sample type, the data here suggests the two plaque sites indicate compositional differences in microbiota. While there is little evidence to support this within the canids, studies in the human field show mixed findings, with some indicating similar bacterial compositions (Sakellari et al. 2001, Mayanagi et al. 2004, Haffajee et al. 2008, Papaioannou et al. 2009, Schaumann et al. 2014) whilst other studies observed differences (Riviere et al. 1992, Ximenez-Fyvie et al. 2000b, Ximenez-Fyvie et al. 2000a, Daniluk et al. 2006, Preza et al. 2009, He et al. 2012) in the taxa and/or their respective proportions. These observations also highlight the potential impact that changes in taxonomic assignments can have on the meta-analysis of studies produced at different times. This issue is likely to become more profound as, for example, interest grows towards the consideration of other oral niches in addition to dental plaque. Therefore, a potential investment to update the associated databases, perhaps periodically in the future, could prove invaluable (Dewhirst et al. 2012, Dewhirst et al. 2015).

Across the supragingival plaque samples, the predominant phyla observed were the Proteobacteria, Firmicutes and Bacteroidetes. This is consistent with the major phyla reported for canine subgingival plaque (Davis et al. 2013, Sturgeon et al. 2013, Holcombe et al. 2014, Wallis et al. 2015). The proportions of phyla varied between the sample groups. Proteobacteria were significantly higher in both test phase groups. This finding reflects another observation relating to the most abundant taxa, a novel Escherichia-Shigella species, representing 28.7% of the total sequence reads. While previous studies on canine microbiota have often isolated P. cangingivalis as the most dominant taxa (Davis et al. 2013, Wallis et al. 2015), this was listed as the 3^{rd} most dominant taxa here (6.27%). Coprophagia provides the clearest explanation for both findings since these taxa typically inhabit the gastrointestinal tract (Schmitz and Suchodolski 2016); the present study did not incorporate a coprophagia management regime. The bacteria associated with environmental factors are also more likely to be represented in supragingival plaque than subgingival plaque. Other phyla differences included Firmicutes being more abundant and Bacteroidetes less abundant in the start pre-test phase samples compared to the end pre-test phase samples. The trends in these majority phyla are consistent with the observations made by (Davis et al. 2013), supporting a more disease orientated plaque microbiome prior to the study test phases. The indications therefore suggest the initial D&P and tooth brushing procedures in the pre-test phase were effective in transitioning the microbiota towards a more healthy composition before beginning the first test phase of the study.

Interestingly, index values for microbial diversity were not able to significantly distinguish the test phase chew and no chew sample groups. Despite our suggested hypothesis regarding differences in biofilm colonisation which leads to mature development without provision of a daily oral care chew versus a relatively early biofilm status, this suggests the 28-day timeframe for the test phase was insufficient to demonstrate a differential result with this parameter. But since far more granularity is provided the invaluable insights into abundances changes for particular bacterial species, diversity parameters alone cannot be a good indicator of health status. Trends from the human literature have shown confidence in the measure with increased bacterial diversity associated with periodontal disease. In contrast, those from within the expanding canine oral research arena have seen mixed conclusions. While Davis et al. 2013 showed the richness of microbiota profiles was significantly higher for mild periodontitis compared to health and for mild periodontitis compared to gingivitis, Wallis et al. 2015 found the parameter did not significantly differ with the development of periodontitis.

The use of clustering techniques illustrated that OTU sample composition was fairly discrete between the plaque sample groups between phases. The chew and no chew groups indicated some commonality in their respective OTU profiles but each also demonstrated unique sample associated microbiota profiles. The commonality most likely relates to primary coloniser species of the developing biofilm community which immediately attach to the tooth surface following the D&P treatments. The pre-test phase groups indicate two very different microbiota profiles; this suggests the D&P followed by two weeks tooth brushing was sufficient to completely change the plaque microbiota profiles, with no reflection on the ‘original’ start-pre-test phase state.

Evaluation of the plaque sample groups at the OTU level indicated a large number of taxa which significantly differed for each pairwise comparison. Taking in to consideration the test phase chew and no chew samples 22 OTUs showed statistically significant differences. Of these, the abundance of 6 health associated OTUs increased while only 3 disease associated OTUs increased with the oral care chew compared to no chew. Actinomyces, previously highlighted by (Davis et al. 2013) to be associated with mild periodontitis, was identified amongst the disease (weak) associated taxa and indicated substantial changes of at least four-fold. Conversely, the abundance of only one health associated OTUs increased and eight disease associated OTUs increased when comparing no chew to chew. The health associated OTU, Desulfovibrio sp. COT-070 (OTU #550), a genus previously not shown to be significantly associated with either health state, showed increased abundance by greater than six-fold. Based on the knowledge that oral care chews help to maintain low plaque and calculus accumulation, we hypothesize that the bacteria associated with these biofilm communities remain stable and towards early colonizer species (Holcombe et al., 2014). This is supported by the observation of an increase in abundance of two primary colonizer species, Corynebacterium mustelae (OTU #8491) and Peptostreptococcaceae bacterium FOT-054 (OTU #8712) by at least two-fold with chew compared to no chew (Holcombe et al. 2014). Further, although Peptostreptococcaceae species have previously found to be associated with periodontal disease, this is completely species dependent.

This example provided that oral chews, such as the one tested in this study, have a positive influence on supragingival plaque microbiota and that bacteria can be employed as biomarkers of dental health rather than solely relying on the quantification of plaque and calculus.

### Example 2: 28 Day Feeding Trial (Daily Oral Care Chews)

This example describes the effects of daily oral care chews included in a diet regimen (28 days) in modulating the canine oral microbiota.

Study Design

Eighteen (18) dogs participated in the study. The study cohort included small (from 5 kg to 10 kg) and medium (from 10 kg to 15 kg) sized dogs: eight (8) Aussie Terriers and ten (10) Beagles. The Aussie Terriers consisted of one (1) male and seven (7) females aged from 1 year to 4.5 years and weighing from 6.3 kg to 8.7 kg. The Beagles consisted of three (3) males and seven (7) females aged from 1 year to 4 years and weighing from 10.0 kg to 15.4 kg.

A balanced 3×3 Latin Square designed study was undertaken in which one of three dietary regimes were fed over three 28-day phases: two of the dietary regimes were (1) commercially available main meal diet+oral care chew B; and (2) commercially available main meal diet alone (control). The commercially available main meal diet comprised a complete and balanced dry food, ADVANCE™ Adult Toy/Small Breed Dry Dog Food (Chicken), formulated to meet AAFCO nutritional requirements for adult dogs. The molecular biology assessments of canine dental plaque were only conducted for the second two dietary regimes and the oral care chew tested and reported for this study comprised a commercially available daily (B) feed treat (GREENIES™ Original (Canine Dental Chews)). Daily intake of food was based on individual energy requirements to maintain bodyweight. For dogs receiving either of the test products, the amount of main meal diet was adjusted to compensate for the energy content of the former.

Prior to the initiation of each 28-day test phase, the dogs underwent a scale and polish (S&P) of their teeth under anaesthesia. For anaesthesia, methadone (0.4 mg/kg) and acepromazine (0.04 mg/kg) were administered as pre-medication by subcutaneous injection. Anaesthesia was induced via intravenous Alfaxan® and maintained by inhalation with oxygen/isoflurane via a chuffed endotracheal tube. The dogs were monitored by a dedicated veterinary nurse throughout anesthesia during which standard procedures were followed and vital statistics recorded.

Prior to S&P, a ‘baseline’ supragingival plaque was collected from the buccal surface of the maxillary and mandibular teeth using a CytoSoft™ cytology brush (Medical Packaging Corporation). Two samples were collected, one from the right and the other from the left hand side of the oral cavity, using separate swabs. The individual plaque samples were immediately placed into 1.5 mL Tris-EDTA (TE, 10 mM Tris-1mM EDTA) buffer (pH 8.0) in 2 mL cryo tubes, with the bristle end of the swab submerged in the buffer, and the plastic swab handles removed. Samples were temporarily stored at −20° C. for up to 36 hours before transferring to longer term storage at −80° C. Upon completion of the study, the plaque samples were transported on dry ice to a center for processing.

During the test phases, all dogs were fed the main meal diet once daily in the afternoon. Dogs receiving chews were offered them after consuming the main meal. The dogs were fed in individual pens and instances of refusal of either the main meal diet and/or chew were weighted and recorded.

DNA Extraction

DNA was extracted from the plaque samples using a Masterpure™ Gram positive DNA purification kit according to the manufacturer's instructions with the addition of an overnight lysis (EpiCentre, catalogue #MGP04100). Plaque samples were centrifuged at 5000×g for 10 minutes and the bacterial pellet re-suspended in 150 μl TE buffer by vortexing. Ready-Lyse™ Lysozyme Solution (1 μl; Epicentre, catalogue #R1804M) was added to the bacterial suspension which was then incubated at 37° C. for 18 hours. Following DNA extraction the DNA pellet was re-suspended in TE buffer (10 mM Tris-Cl and 0.5 mM pH 9.0 EDTA). The quantity of DNA was determined using a Qubit® dsDNA High Sensitivity Assay Kit (Thermo Fisher Scientific Inc.).

Quantitative PCR (qPCR) analyses

A qPCR assay developed against the 16S rRNA gene of Peptostreptococcaceae XIII [G-1] sp. (COT-030) and a universal qPCR assay (UniB) designed and validated in-house were used to assess levels of the species.

Each individual 10 μl quantitative PCR (qPCR) reaction contained: 5 μL Applied Biosytems Gene Expression Taqman MasterMix (Applied Biosystems, USA), 0.5 μL 20× concentrated assay, 1 μL 1:10 dilution of DNA and 3.5 μL nuclease-free water. Each assay contained a final concentration of 900 nM of each primer and 250 nM of each qPCR probe per reaction. Experiments were performed in triplicate. Positive and negative controls, also included in triplicate, were the M13 purified amplicon of the species clone (CN030) at 0.001 ng/μl and nuclease-free water, respectively. Data were collected on an ABI QuantStudio 7 Flex Real-Time PCR System (Applied Biosystems, USA) and analyzed using GenEx software (MultiD, Sweden).

COT-030 normalised to UniB relative to nothing was calculated by performing the following equation on the mean, efficiency corrected Cq value for each sample and COT030 assay: 2\hat{ }^{-(mean COT030 Cq value-mean UniB Cq value)}.

Statistical Analysis

COT-030 normalised to UniB relative to nothing values were represented as mean +/−95% Confidence Interval (Ci).

A linear mixed model was applied on the log 10 transformed parameter with dietary regimes, phases, two-way interactions and three-way interactions as fixed effects and dog as random effect. Normality and homogeneity of residuals were tested with Shapiro and Bartlett tests, a variance correction was applied because the conditions of Bartlett were not met. A Tukey post hoc comparison was used to compare dietary regimes and phases if relevant.

All analyses were performed using R version 3.6.1.

### Results

Assessment of Variable Study Parameters

Since a balanced 3×3 Latin Square design was adopted for the study, the preliminary analysis of the data set out to determine whether the dietary regimes were impacted by the phased approach. The findings from this preliminary investigation are shown in Table 6.

TABLE 6
Analysis of Variants

| Contrasts | p-value |
|---|---|
| Dietary Regime | <0.001 |
| Phase | 0.046 |
| Dietary Regime:Phase | 0.184 |

The resulting p values indicate significant differences in dietary regime and phase when tested as single, individual factors. A significant difference was not observed in the interactions between dietary regime and phase. This suggests dietary regime differences are not dependent on the study design.

Effect of Dietary Regimes

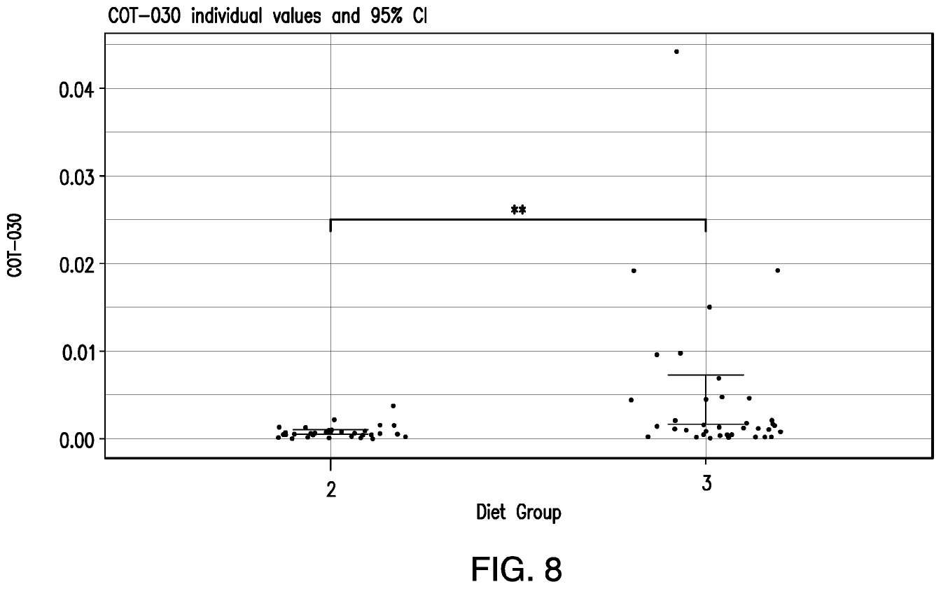

The qPCR results for the dietary regimes tested in the study, expressed as COT-030 normalised to UniB relative to nothing, were then subject to a pairwise comparison, shown in FIG. 8. The contrast performed between the test dietary regime and control revealed a significant difference between the groups. The oral care chew B, represented by dietary regimes 2 (FIG. 8) was found to be significantly different from the control (Dietary Regime 3). The resulting p-values was p=0.001.

### DISCUSSION

For many years the efficacy of canine dental chews has been clinically proven via measurements of plaque and calculus. These factors are well established in their association with periodontal disease and their quantification therefore offers a means to understand the effectiveness of preventative oral health measures.

High throughput sequencing has enabled the association of bacterial species abundant in canine and feline dental plaque with specific health states. For canine periodontal disease, Peptostreptococcaceae XIII [G-1] sp. (COT-030) has been shown to be well correlated with the early stages of disease, thus offering a good discriminatory biological indicator.

Through quantification of Peptostreptococcaceae XIII [G-1] sp. (COT-030) via molecular biology based approaches, we have demonstrated the efficacy of an oral care chew with a daily feeding regime. By showing a significant reduction in the level of the disease associated species in supragingival plaque after a 28 day feeding period, compared to the control, existing product efficacy data has been supplemented with a novel microbial measure.

### REFERENCES

Altschul, S. F., W. Gish, W. Miller, E. W. Myers and D. J. Lipman (1990). “Basic local alignment search tool.” J Mol Biol 215(3): 403-410.
Bates, D., M. Mächler, B. Bolker and S. Walker (2015). “Fitting Linear Mixed-Effects Models Using lme4.” 2015 67(1): 48.
Benjamini, Y. and Y. Hochberg (1995). “Controlling the False Discovery Rate: A Practical and Powerful Approach to Multiple Testing.” Journal of the Royal Statistical Society. Series B (Methodological) 57(1): 289-300.
Brown, W. Y. and P. McGenity (2005). “Effective periodontal disease control using dental hygiene chews.” J Vet Dent 22(1): 16-19.
Caporaso, J. G., J. Kuczynski, J. Stombaugh, K. Bittinger, F. D. Bushman, E. K. Costello, N. Fierer, A. G. Pena, J. K. Goodrich, J. I. Gordon, G. A. Huttley, S. T. Kelley, D. Knights, J. E. Koenig, R. E. Ley, C. A. Lozupone, D. McDonald, B. D. Muegge, M. Pirrung, J. Reeder, J. R. Sevinsky, P. J. Turnbaugh, W. A. Walters, J. Widmann, T. Yatsunenko, J. Zaneveld and R. Knight (2010). “QIIME allows analysis of high-throughput community sequencing data.” Nat Methods 7(5): 335-336.
Clarke, D. E., M. Kelman and N. Perkins (2011). “Effectiveness of a vegetable dental chew on periodontal disease parameters in toy breed dogs.” J Vet Dent 28(4): 230-235. Daniluk, T., G. Tokajuk, D. Cylwik-Rokicka, D. Rozkiewicz, M. L. Zaremba and W. Stokowska (2006). “Aerobic and anaerobic bacteria in subgingival and supragingival plaques of adult patients with periodontal disease.” Adv Med Sci 51 Suppl 1: 81-85.
Davis, E. M. (2016). “Gene Sequence Analyses of the Healthy Oral Microbiome in Humans and Companion Animals.” J Vet Dent 33(2): 97-107.
Davis, I. J., C. Wallis, O. Deusch, A. Colyer, L. Milella, N. Loman and S. Harris (2013). “A cross-sectional survey of bacterial species in plaque from client owned dogs with healthy gingiva, gingivitis or mild periodontitis.” PLoS One 8(12): e83158.
Dewhirst, F. E., E. A. Klein, M. L. Bennett, J. M. Croft, S. J. Harris and Z. V. Marshall-Jones (2015). “The feline oral microbiome: a provisional 16S rRNA gene based taxonomy with full-length reference sequences.” Vet Microbiol 175(2-4): 294-303.
Dewhirst, F. E., E. A. Klein, E. C. Thompson, J. M. Blanton, T. Chen, L. Milella, C. M. Buckley, I. J. Davis, M. L. Bennett and Z. V. Marshall-Jones (2012). “The canine oral microbiome.” PLoS One 7(4): e36067.
Edgar, R. C. (2010). “Search and clustering orders of magnitude faster than BLAST.” Bioinformatics 26(19): 2460-2461.
Elliott, D. R., M. Wilson, C. M. Buckley and D. A. Spratt (2005). “Cultivable oral microbiota of domestic dogs.” J Clin Microbiol 43(11): 5470-5476.
Fadrosh, D. W., B. Ma, P. Gajer, N. Sengamalay, S. Ott, R. M. Brotman and J. Ravel (2014). “An improved dual-indexing approach for multiplexed 16S rRNA gene sequencing on the Illumina MiSeq platform.” Microbiome 2(1): 6.
Gad, T. (1968). “Periodontal disease in dogs. 1. Clinical investigations.” J Periodontal Res 3(4): 268-272.
Gorrel, C. and T. L. Bierer (1999). “Long-term effects of a dental hygiene chew on the periodontal health of dogs.” J Vet Dent 16(3): 109-113.
Gorrel, C., J. Warrick and T. L. Bierer (1999). “Effect of a new dental hygiene chew on periodontal health in dogs.” J Vet Dent 16(2): 77-81.
Haffajee, A. D., S. S. Socransky, M. R. Patel and X. Song (2008). “Microbial complexes in supragingival plaque.” Oral Microbiol Immunol 23(3): 196-205.
Harvey, C., L. Serfilippi and D. Barnvos (2015). “Effect of Frequency of Brushing Teeth on Plaque and Calculus Accumulation, and Gingivitis in Dogs.” J Vet Dent 32(1): 16-21.
Harvey, C. E., F. S. Shofer and L. Laster (1994). “Association of age and body weight with periodontal disease in North American dogs.” J Vet Dent 11(3): 94-105.
He, J., W. Huang, Z. Pan, H. Cui, G. Qi, X. Zhou and H. Chen (2012). “Quantitative analysis of microbiota in saliva, supragingival, and subgingival plaque of Chinese adults with chronic periodontitis.” Clin Oral Investig 16(6): 1579-1588.
Hennet, P., E. Servet and C. Venet (2006). “Effectiveness of an oral hygiene chew to reduce dental deposits in small breed dogs.” J Vet Dent 23(1): 6-12.
Hoffmann, T. and P. Gaengler (1996). “Epidemiology of periodontal disease in poodles.” 1 Small Anim Pract 37(7): 309-316.
Holcombe, L. J., N. Patel, A. Colyer, O. Deusch, C. O'Flynn and S. Harris (2014). “Early canine plaque biofilms: characterization of key bacterial interactions involved in initial colonization of enamel.” PLoS One 9(12): e113744.
Hothorn, T., F. Bretz and P. Westfall (2008). “Simultaneous Inference in General Parametric Models.” Biometrical Journal 50(3): 346-363.
Keijser, B. J. F., T. J. van den Broek, D. E. Slot, L. van Twillert, J. Kool, C. Thabuis, M. Ossendrijver, F. A. van der Weijden and R. C. Montijn (2018). “The Impact of Maltitol-Sweetened Chewing Gum on the Dental Plaque Biofilm Microbiota Composition.” Front Miergbil 9:381.
Knaus, J. (2013). Snowfall: Easier cluster computing (based on snow).
Kortegaard, H. E., T. Eriksen and V. Baelum (2008). “Periodontal disease in research beagle dogs—an epidemiological study.” J Small Anim Pract 49(12): 610-616.
Kyllar, M. and K. Witter (2005). “Prevalence of dental disorders in pet dogs.” Veterinary Medicine—Czech 50(11): 496-505.
Lund, E. M., P. J. Armstrong, C. A. Kirk, L. M. Kolar and J. S. Klausner (1999). “Health status and population characteristics of dogs and cats examined at private veterinary practices in the United States.” J Am Vet Med Assoc 214(9): 1336-1341.
Magoc, T. and S. L. Salzberg (2011). “FLASH: fast length adjustment of short reads to improve genome assemblies.” Bioinformatics 27(21): 2957-2963.
Mayanagi, G., T. Sato, H. Shimauchi and N. Takahashi (2004). “Detection frequency of periodontitis-associated bacteria by polymerase chain reaction in subgingival and supragingival plaque of periodontitis and healthy subjects.” Oral Microbiol Immunol 19(6): 379-385.
Murdoch, D. and E. D. Chow (2013). ellipse: Functions for Drawing Ellipses and Ellipse-Like Confidence Regions.
O. Neill, D. G., D. B. Church, P. D. McGreevy, P. C. Thomson and D. C. Brodbelt (2014). “Prevalence of disorders recorded in dogs attending primary-care veterinary practices in England.” PLoS One 9(3): e90501.
Papaioannou, W., S. Gizani, A. D. Haffajee, M. Quirynen, E. Mamai-Homata and L. Papagiannoulis (2009). “The microbiota on different oral surfaces in healthy children.” Oral Microbiol Immunol 24(3): 183-189.
Preza, D., I. Olsen, T. Willumsen, B. Grinde and B. J. Paster (2009). “Diversity and site-specificity of the oral microflora in the elderly.” Eur J Clin Microbiol Infect Dis 28(9): 1033-1040.
Pruesse, E., C. Quast, K. Knittel, B. M. Fuchs, W. Ludwig, J. Peplies and F. O. Glockner (2007). “SILVA: a comprehensive online resource for quality checked and aligned ribosomal RNA sequence data compatible with ARB.” Nucleic Acids Res 35(21): 7188-7196.
Quest, B. W. (2013). “Oral health benefits of a daily dental chew in dogs.” J Vet Dent 30(2): 84-87.
Riggio, M. P., A. Lennon, D. J. Taylor and D. Bennett (2011). “Molecular identification of bacteria associated with canine periodontal disease.” Vet Microbiol 150(3-4): 394-400.
Riviere, G. R., K. S. Elliot, D. F. Adams, L. G. Simonson, L. B. Forgas, A. M. Nilius and S. A. Lukehart (1992). “Relative proportions of pathogen-related oral spirochetes (PROS) and Treponema denticola in supragingival and subgingival plaque from patients with periodontitis.” J Periodontol 63(2): 131-136.
Rohart, F., B. Gautier, A. Singh and K. A. Le Cao (2017). “mixOmics: An R package for 'omics feature selection and multiple data integration.” PLoS Comput Biol 13(11): e1005752.
Sakellari, D., G. Belibasakis, T. Chadjipadelis, K. Arapostathis and A. Konstantinidis (2001). “Supragingival and subgingival microbiota of adult patients with Down's syndrome. Changes after periodontal treatment.” Oral Microbiol Immunol 16(6): 376-382.
Schaumann, S., I. Staufenbiel, R. Scherer, M. Schilhabel, A. Winkel, S. N. Stumpp, J. Eberhard and M. Stiesch (2014). “Pyrosequencing of supra- and subgingival biofilms from inflamed peri-implant and periodontal sites.” BMC Oral Health 14: 157.
Schmieder, R., Y. W. Lim, F. Rohwer and R. Edwards (2010). “TagCleaner: Identification and removal of tag sequences from genomic and metagenomic datasets.” BMC Bioinformatics 11: 341.
Schmitz, S. and J. Suchodolski (2016). “Understanding the canine intestinal microbiota and its modification by pro-, pre- and synbiotics—what is the evidence?” Veterinary Medicine and Science 2(2): 71-94.
Sturgeon, A., J. W. Stull, M. C. Costa and J. S. Weese (2013). “Metagenomic analysis of the canine oral cavity as revealed by high-throughput pyrosequencing of the 16S rRNA gene.” Veterinary MicrobiolQgy 162(2): 891-898.
Team, R. C. (2017). R: A language and environment for statistical computing. Available from: www.R-project.org.
Wallis, C., M. Marshall, A. Colyer, C. O'Flynn, O. Deusch and S. Harris (2015). “A longitudinal assessment of changes in bacterial community composition associated with the development of periodontal disease in dogs.” Vet Microbiol 181(3-4): 271-282.
Wickham, H. (2009). ggplot2: Elegant Graphics for Data Analysis. New York, Springer-Verlag.
Ximenez-Fyvie, L. A., A. D. Haffajee and S. S. Socransky (2000a). “Comparison of the microbiota of supra- and subgingival plaque in health and periodontitis.” J Clin Periodontol 27(9): 648-657.
Ximenez-Fyvie, L. A., A. D. Haffajee and S. S. Socransky (2000b). “Microbial composition of supra- and subgingival plaque in subjects with adult periodontitis.” J Clin Periodontol 27(10): 722-732.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1-22. (canceled)

23. A method of modulating an oral microbiota in a dog, comprising administering an oral chew in an amount effective to improve the oral health of the dog.

24. The method of claim 23, wherein the dog is administered the oral chew daily, twice weekly, weekly or fortnightly.

25. The method of claim 23, wherein the dog is administered the oral chew over a period of 3 days a year, for the life of the dog.

26. The method of claim 23, wherein the dog is administered the oral chew at least 5, 8, 10, 13, 15, 18, 20, 23, 25, 28, 35 or 42 times.

27. The method of claim 23, further comprising cleaning the mouth of the dog prior to administering the oral chew.

28. The method of claim 27, wherein the mouth of the dog is cleaned prior to administering any oral chew.

29. The method of claim 23, wherein the dog is from a medium, large or giant breed.

30. The method of claim 23, wherein the oral microbiota is modulated by increasing the number of bacterial species associated with good oral health, or the prevalence or relative proportion of bacteria from bacterial species associated with good oral health, compared to an expected microbiota.

31. The method of claim 30, wherein modulation of the oral microbiota comprises increasing the prevalence of at least one of Prevotella sp. COT-282, Propionibacterium sp. COT-296, Catonella sp. COT-257, Peptostreptococcaceae bacterium FOT-054 and Corynebacterium mustelae.

32. The method of claim 23, wherein the oral microbiota is modulated by decreasing the number of bacterial species associated with poor oral health or with disease, or the prevalence or relative proportion of bacteria from bacterial species associated with poor oral health or disease, compared to an expected microbiota.

33. The method of claim 32, wherein modulating the oral microbiota comprises decreasing the prevalence of at least one of Fretibacterium sp. FOT-218, Neisseria canis, Anaerovorax sp. COT-125, Peptostreptococcaceae bacterium COT-030, Pelistega sp. COT-267, Bacteroidia bacterium COT-387, Desulfomicrobium orale and Helococcus sp. FOT-023.

34. The method of claim 23, wherein the improved oral health of the dog is a reduction in periodontal disease or oral malodour.

35. The method of claim 23, wherein the dog is from a toy or small breed.


SEQUENCE LISTING (24 sequences)

SEQ ID NO: 1 - DNA, 20 bp, Artificial Sequence
  Description of Artificial Sequence Synthetic oligonucleotide
ggactachvgggtwtctaat

SEQ ID NO: 2 - DNA, 20 bp, Artificial Sequence
  Description of Artificial Sequence Synthetic oligonucleotide
ctgctgcctcccgtaggagt

SEQ ID NO: 3 - DNA, 429 bp, Unknown
  Description of Unknown unclassified Klebsiella [novel] sequence
cctgtttgctccccacgctttcgcacctgagcgtcagtctttgtccagggggccgccttcgccaccggtattcctccagatctctacgcatttcaccgctacacctggaattctacccccctctacaagactctagcctgccagtttcgaatgcagttcccaggttgagcccggggatttcacatccgacttgacagaccgcctgcgtgcgctttacgcccagtaattccgattaacgcttgcaccctccgtattaccgcggctgctggcacggagttagccggtgcttcttctgcgggtaacgtcaatcgacaaggttattaaccttatcgccttcctccccgctgaaagtactttacaacccgaaggccttcttcatacacgcggcatggctgcatcaggcttgcgcccattgtgcaatattcccca

SEQ ID NO: 4 - DNA, 410 bp, Propionibacterium sp.
  COT-296
gcctgttcgctccccacgctttcgcttctcagcgtcaggaaaggtccagagaaccgccttcgccaccggtgttcctcctgatatctgcgcattccaccgctccaccaggaattccgttctcccctacctccctcaagtcagcccgtatcgaaagcaagctcagagttaagccctgagttttcactcccgacgcgacaaaccgcctacaagctctttacgcccaataaatccggacaacgctcgcaccctacgtatcaccgcggctgctggcacgtagttagccggtgcttcttctgtcggtaccgtcactcacgcttcgtcccgactgaaagcggtttacaacccgaaggccgtcatcccgcacgcggcgttgctgcatcaggcttccgcccattgtgcaatattcccca

SEQ ID NO: 5 - DNA, 404 bp, Catonella sp.
  COT-257
cctgtttgctacccacgctttcgagcctcagcgtcagttttggtccagcaagccgccttcgccaccggtgttcttcctaatatctaagcatttcaccgctacactaggaattccgcttgcctctcccatactcaagcctaacagttttgggagcagtctcggggttgagccccgagcttccactctcaacttgaaaggccgcctgcgctccctttacacccagtaaatccggataacgcttgccccctacgtattaccgcggctgctggcacgtagttagccggggcttcttagtcaggtaccgtcatcatcttccctgctgatagagctttacataccgaaatacttcttcactcacgcggcgtcgctgcatcagagtttcctccattgtgcaatatccccca

SEQ ID NO: 6 - DNA, 416 bp, Corynebacterium mustelae
cctgttcgctccccatgctttcgctcctcagcgtcagttactgcccagagacctgccttcgccatcggtgttcctcctgatatctgcgcattccaccgctacaccaggaattccagtctcccctacagcactccagttatgcccgtatcgcctgcaaccccgaagttaagccccggtatttcacagacgacgcaacaaaccacctacgagctctttacgcccagtaattccggacaacgctcgcaccctacgtattaccgcggctgctggcacgtagttagccggtgcttcttctacaggtaccgtcaccttaaaaaaggcttcgtccctaccgaaagaggtttacaacccgaaggccgtcatcccccacgcggcgtcgctgcatcaggcttgcgcccattgtgcaatattcccca

SEQ ID NO: 7 - DNA, 424 bp, Prevotella sp.
  COT-282
cctgtttgatacccacactttcgagcctcagcgtcagttgtgctcccggcatatgccttcgcgatcggagttcttcgtaatatctaagcatttcaccgctacactacgaattccaatgccgctgcgcacactcaagacaaccagtatcaactgcaattttaaggttgagcctcaaactttcacagctgacttaatcatccgcctacgctccctttaaacccaataaatccggataacgcccgaaccttccgtattaccgcggctgctggcacggaattagccggtcctttttcttacggtacttgcaagacaccacacgtggcgttttttacccccgtataaaagcagtttacaacccagagggcagtcttcctgcacgctacttggctggttcagactctcgtccattgaccaatattcctca

SEQ ID NO: 8 - DNA, 406 bp, Unknown
  Description of Unknown TM7 sp. COT-305 sequence
cccgttcgctccccacgctttcgtgccttagcgtcagaaatggtccagtaacctgcctacgccattggtgttccttctaatatctacggatttcactcctacactagaaattccagttacctctaccactctcgagtttagcagtttgaataatagtctgtatggttgagccaccaggtttcactattcacttactaaaccgcctacgcaactctttacgcccagtcactccggataatgcttgcaccctacgtatgaccgcggctgctggcacgtagttagccggtgcttattcatgagttaccgtcatattcttcactcataaaagaagtttacaacccgaaggccttcatccttcacgcggcgttgctccatcaggctttcgcccattgtggaagattcctca

SEQ ID NO: 9 - DNA, 403 bp, Unknown
  Description of Unknown Parvimonas sp. sequence
  COT-101
cctgtttgctccccacgctttcgtacctgagcgtcagtaaaagtccagaaagtcgccttcgccaccggtattcctcctaatatctacgcatttcaccgctacactaggaattccactttcctctccttcactcaagccttccagtttcaagtgcttaatgaggttaagcctcacgctttcacacctgacttaaaaggccgcctacgtaccctttacgcccaataattccggacaacgctcgccccatacgtattaccgcggctgctggcacgtatttagccggggcttcctcctatgataccgtcattatcttctcataggacagagctttacgactcgaaagccttcttcgctcacgcggcgtcgctgcatcagggtttcccccattgtgcaatattcccca

SEQ ID NO: 10 - DNA, 421 bp, Unknown
  Description of Unknown unclassified Actinomyces [novel 3] sequence
cctgttcgctccccacgctttcgctcctcagcgtcagtaacggcccagagacccgccttcgccaccggtgttcctcctgatatctgcgcattccaccgctacaccaggaattccagtctcccctaccgcactcaagccagcccgtacccaccgcaagcccggagttaagccccgggttttcacggcagacgcgacaagccgcctacaagccctttacgcccaataattccggacaacgctcgcgccctacgtattaccgcggctgctggcacgtagttagccggcgcttctttacccactaccctcaactagaacaaaaactagccttgaccatgagcgaaagaggtttacaacccgaaggccgtcatccctcacgcggcgttgctgcatcaggcttgcgcccattgtgcaatattcccca

SEQ ID NO: 11 - DNA, 430 bp, Unknown
  Description of Unknown unclassified Treponema [novel 2] sequence
cctgtttgctccccgcaccttcgcatatcagcgtcaatcatcggccagaaacccgccttcgccaccggtgttcttccaaatatctacagattccacccctacacttggaattccggtttcccctccgtgattcaagttaagcagtacccaatgcagtttacgagttaagctcgtagatttcacatcaggcttacctaaccgcctacatgccctttacgcccaataattccgaacaacgcttggggcttacgtgttaccgcggctgctggcacgtaattagccgccccttattcgcatgattaccgtcatcagataggcattccctcctatccttattcttcatctgcaaaagaactttacaacctttcggccttcatcgttcacgcggcgtcgctccgtcagactttcgtccattgcggaagattcttag

SEQ ID NO: 12 - DNA, 403 bp, Unknown
  Description of Unknown Bacterial sequence
ccctttcgctccccacgctttcgtctctcagtgtcagtagtgttccagcaagctgccttcgcttttggtattctggtatgtatcaacggattgcacccctactcataccgttctgcttgcctctcccacactcaagttatatggttttccgcgcaatccagggttgagccctggagtttcacacgaaacctttataaccacctacagacgctttacgcccagtaattccggataacgcttggggccctcgtattaccgcggctgctggcacgaagtttgccgccccttattcctctcgtaccgtcattatcttccgagagaaaagaagtttacaccaacaaaggcttcatccttcacgcggtgtcgctccatcaggctttcgcccattgtggaagattcctca

SEQ ID NO: 13 - DNA, 429 bp, Unknown
  Description of Unknown Desulfovibrio sp. sequence
  COT-070
cctgtttgctccccacgctttcgcacctcagcgtcaataccggtccaggtggccgccttcgccactgatgttcctccagatatctacggatttcactcctacacctggaattccgccaccctctcccggattcaagtcacgcagtatcaaaggcagttccacggttgagccgtgggatttcacccctgacttacatgacagcctacgtgcgctttacgcccagtaattccgattaacgctcgcaccctccgtattaccgcggctgctggcacggagttagccggtgcttcctttgaaggtaccgtcaatacacccctgattggcagagtgcaccttcttcccttccgacagaggtttacgatccgaaaaccttcatccctcacgcggcgtcgctgcgtcaggctttcgcccattgcgcaatattcccca

SEQ ID NO: 14 - DNA, 406 bp, Unknown
  Description of Unknown Fretibacterium sp. sequence
  FOT-218
cctgtttgctccccacgctttcgcacctgagcgtcagttaccgtccagcaagtcgccttcgccaccgatgttcttcccaatatctacgcatttcaccgctacactgggaattccacttgcctctccggtactccagcacctcagtctcaactgcataacacggttaagccgcatcctttaacagctgacttgaagcacagcctgcgtgccctttacgcccagtaattccggacaacgctcgccccctacgtattaccgcggctgctggcacgtagttagccggggcttattcatgtggtaccgtcactctcttcttcccacataaaagaactttacgacccgaaggccttcttcgttcacgcggcgtcgctgggtcaggattcctcccattgcccaatattcccca

SEQ ID NO: 15 - DNA, 403 bp, Helcococcus sp.
  FOT-023
cctgtttgctccccacgctttcgtacctcagcgtcagttagattccagaaagtcgccttcgccaccggtattcctccaaatatctacgcatttcaccgctacacttggaattccactttcccctcatctactcaagttatccagtttccacaccttacattggttgagccaatgccttttaatatggacttaaataaccgcctacgtaccctttacgcccaataattccggacaacgctcgccccatacgtattaccgcggctgctggcacgtatttagccggggctttcttcttggttactgtcattatcttcaccaaggacagaactttacaacccgaaggccttcttcgttcacgcggcgtcgctgcatcagggtttcccccattgtgcaaaattcccca

SEQ ID NO: 16 - DNA, 404 bp, Unknown
  Description of Unknown unclassified Clostridium [novel] sequence
cctgtttgctccccacgctttcgagcctcagcgtcagttacagtccagagagtcgccttcgccactggtgttcttcctaatctctacgcatttcaccgctacactaggaattccactctcctctcctgcactctagataaccagtttggaatgcagcacccaagttgagcccgggtatttcacatcccacttaatcatccgcctacgctccctttacgcccagtaaatccggataacgctcgccacctacgtattaccgcggctgctggcacgtagttagccgtggcttcctccttgggtaccgtcattatcttccccaaagacagagctttacgatccgaaaaccttcatcactcacgcggcgttgctgcatcagggtttcccccattgtgcaatattcccca

SEQ ID NO: 17 - DNA, 430 bp, Desulfomicrobium orale
cctgtttgctccccacactttcgcacctcagcgtcaatacctgtccaggcggccgccttcgccaccggtgttcctcctgatatctacggatttcactcctacaccaggaattccgccgccctctccaggattcgagccccgcagtttcaagtgcagttccacggttgagccgtgggatttcacacctgacttacaaggccgcctacgtgcgctttacgcccagtaattccgaataacgcttgcaccctccgtattaccgcggctgctggcacggagttagccggtgcttcctttgaaggtaccgtcaaaatgcgggcctattggacccgcatcacttcttcccttctgacagaggtttacgatccgaaaaccttcatccctcacacggcgttgctgcgtcaggctttcgcccattgcgcaatattcccca

SEQ ID NO: 18 - DNA, 406 bp, Anaerovorax sp.
  COT-125
cctgtttgctacccacgctttcgtgcctcagtgtcagttacagtccagaaagccgccttcgccaccggtgttcctcctaatatctacgcatttcaccgctacactaggaattccactttcccctcctgcactcaagctacacagttcgcagggcttacaatggttaagccactgcctttcaccccacgcttatctagccacctacgcactctttacgcccaataattccggataacgctcgccccctacgtattaccgcggctgctggcacgtagttagccggggctttcttgataggtaccgtcacctttttcttccctatcgacagagctttacgacccaaaggccttcttcgctcacgcggcgttgctgcatcaggctttcgcccattgtgcaatattcccca

SEQ ID NO: 19 - DNA, 423 bp, Unknown
  Description of Unknown Bacteroidia bacterium sequence
  COT-387
cctgttcgatacccacgccttcgtgcatcagcgtcaatgaggggctcgcgagatgccttcgcaatcggtgttctgtgtgatatctatgcatttcaccgctacaccacacattcctcccgcggcgccccaattcaagcgcgacagtttcgacggcaaaccgcgcgttgagcgcgaggatttcaccgccgacttgacacgcagcctacgcaccctttaaacccaataaatccggataacgctcgcatcccccgtattaccgcggctgctggcacggagttagccgatgcttattcgaccggtactctcatcgggccaccagtggcccttattgctcccggtcaaaaggagtttaagacccgtagggccgtcgcctccacgcggcatggctggatcaggcttacgcccattgtccaatatccctca

SEQ ID NO: 20 - DNA, 429 bp, Neisseria canis
cctgtttgctacccacgctttcgagcatgaacgtcagtattatcccagggggctgccttcgccatcggtattcctccacatctctacgcatttcactgctacacgtggaattctacccccctctgacatactctagttacccagttcagaacgccgttcccaggttaagcccggggatttcacatcctgcttaagtaaccgtctgcgctcgctttacgcccagtaattccgattaacgctcgcaccctacgtattaccgcggctgctggcacgtagttagccggtgcttattcttacggtaccgtcataacttcagggtattagcccaaagcctttcttcccgtacaaaagtcctttacaacccgaaggccttcttcagacacgcggcatggctggatcagggttccccccattgtccaaaattcccca

SEQ ID NO: 21 - DNA, 429 bp, Pelistega sp.
  COT-267
cctgtttgctccccacgctttcgtgcatgagcgtcagtattatcccagggggctgccttcgccatcggtattcctccacatctctacgcatttcactgctacacgtggaattctacccccctctgacatactctagttcgggagttaaaaatgccgttccaaggttgagccctgggatttcacatctttctttccgaaccgcctgcgcacgctttacgcccagtaattccgattaacgcttgcaccctacgtattaccgcggctgctggcacgtagttagccggtgcttattcttcaggtaccgtcatcacgcaaaggtattaactctgcgcttttcttccctgacaaaagtgctttacaacccgaaggccttcatcgcacacgcgggatggctggatcagggttccccccattgtccaaaattcccca

SEQ ID NO: 22 - DNA, 404 bp, Unknown
  Description of Unknown Bacterial sequence
cctgtttgctccccacgctttcgcgcctcagcgtcagttaatgtccagcaggccgccttcgccactggtgttcctccctatatctacgcatttcaccgctacacagggaattccgcctgcctctccatcactcaagaactacagtttcaagtgcacgctcggggttgagccccgagatttcacacctgacttgcagtcccgcctacacgccctttacacccagtaaatccggataacgcttgccacctacgtattaccgcggctgctggcacgtagttagccgtggcttattcctgaagtaccgtcattatcttccctcagaaaagaagtttacaacccgaaagccttcttccttcacgcggcgttgctgggtcaggcttgcgcccattgcccaatattcccca

SEQ ID NO: 23 - DNA, 408 bp, Unknown
  Description of Unknown Bacterial sequence
ccgttttgctacccacgctttcgagccttagtgtcagacaaggaccagcgaatcgccttcgccactgatgttcctcccaatatcaacgcatttcaccgctccactgggaattccattcgcctcttcctgtctcaattcctgtagtttccaaagctttcccacggttgagccgtggtctttaactaaagacttacagaaacacctacgcatctctatacacccaataaatccggataacgttcgcaccctacgcatcaccgatgcttctggcacgtagttagcaggtgcttattcatatggtaccgtcatctattcttcccatataaaaggagtttacaatccgaagaccgtcatcctccacactgtgtcgctgcgtcagggttgcccccattgcgcaagattcctaat

SEQ ID NO: 24 - DNA, 429 bp, Unknown
  Description of Unknown Bacterial sequence
cctgtttgctccccacgctttcgtgcctcagcgtcagtataggcccagcaagccgccttcgccactggtgttcctccatatatttacgcattttaccgctacacatggaattccacttgcctctacctaactctagtctcccagttttcaaagcgttccaaagttgagctttggatttaaaccttgaacttaaaaaaccgcctacgcaccctttacgcccaataattccggataacgcttgccccctatgtattaccgcggctgctggcacatagttagccggggcttattcatttagtaccgtcaatatcatatcatttcctatacaatatgttcttcctaaataaaagaattttacgtactagaagtatgtcttcattcacgcggtatcgctcggtcagggtttcccccattgccgaagattctcta